(19) United States
(12) Patent Application Publication
(10) Pub. No.: US 2011/0098200 A1
(43) Pub. Date: Apr. 28, 2011

(54) Methods using dsdna to mediate rna interference (rnai)

(75) Inventors: Gregory Martin Arndt, New South Wales (AU); Murray Cairns, New South Wales (AU); Nham Tran, New South Wales (AU); Angela Lai, New South Wales (AU)

(73) Assignee: Johnson & Johnson Research Pty Ltd

(21) Appl. No.: 10/526,475

(22) PCT Filed: Sep. 4, 2003
(86) PCT No.: PCT/AU03/01142

§ 371 Date: Oct. 5, 2005

(30) Foreign Application Priority Data
Sep. 4, 2002 (AU) 2002951224
Mar. 26, 2003 (AU) 2003901418

(51) Int. Cl. C40B 50/06; C12P 19/34; C12N 15/64; C12Q 1/68; C12N 15/63; C12N 5/071

(52) U.S. Cl. 506/26; 435/91.5; 435/91.4; 435/6; 435/320.1; 435/325

(57) ABSTRACT

The present invention provides methods of producing dsDNA molecules that can be used to mediate RNA interference (RNAi). These methods include the production of hairpin DNAs including random sequences, and the use of convergent promoters to co-express sense and antisense RNAs. As such, the invention allows the production of random short hairpin RNA (shRNA) and a small interfering RNA (siRNA) expression libraries for forward genetic screening.

### FIELD OF THE INVENTION

The present invention relates to methods of producing libraries of DNA molecules the transcription of which results in the production of double stranded RNA or hairpin RNA. The present invention further relates to short interfering RNA expression vectors.

### BACKGROUND OF THE INVENTION

The introduction of double stranded RNA (dsRNA) into a range of organisms induces both a potent and specific gene silencing effect. This form of gene suppression by a dsRNA molecule was first observed in Caenorhabditis elegans and given the term RNA interference or RNAi (Fire et al 1998). In an attempt to optimise the use of antisense RNA as a tool for controlling specific gene expression in worms, Fire et al (1998) found that dsRNA was more effective than antisense RNA alone. The dsRNA could be generated in vitro (Fire et al 1998) or in vivo (Tavernarakis et al 2000) and still mediate gene suppression with high specificity. Subsequent studies have shown that dsRNA is an effective inducer of gene silencing in a wide range of eukaryotic organisms and that the mechanism behind this form of gene regulation is most likely conserved throughout evolution (Baulcombe, D. C. (1996) Plant Mol Biol 32(1-2), 79-88; Lohmann, J. U., Endl, I., and Bosch, T. C. (1999) Dev Biol 214(1), 211-4; Ngo, H., Tschudi, C., Gull, K., and Ullu, E. (1998) Proc Natl Acad Sci USA 95(25), 14687-92; Cogoni, C., and Macino, G. (1999) Nature 399(6732), 166-9; Kennerdell, J. R., and Carthew, R. W. (1998) Cell 95(7), 1017-26; Schoppmeier, M., and Damen, W. G. (2001) Dev Genes Evol 211(2), 76-82; Baker, M. W., and Macagno, E. R. (2000) Curr Biol 10(17), 1071-4; Wargelius, A., Ellingsen, S., and Fjose, A. (1999) Biochem Biophys Res Commun 263(1), 156-61).

The molecular mechanism of RNAi has begun to be deciphered using biochemical and genetic approaches in different experimental systems (Hammond, S. M., Caudy, A. A., and Harmon, G. J. (2001) Nat. Rev. Genet. 2, 110-19). Presently, RNAi is postulated to involve both an initiation step and an effector step. During the initiation phase, dsRNA is processed by the RNaseIII family nuclease Dicer to produce 21-23 nucleotide duplex siRNAs (small interfering RNAs). These short stretches of dsRNA carry 2 nucleotide 3′-OH overhangs that contribute to the efficacy of gene silencing (Elbashir, S. M., Lendeckel, W., and Tuschl, T. (2001) Genes & Dev 15:188-200). In the effector phase, these siRNAs are incorporated into a multiprotein complex called RISC (RNA-induced silencing complex) that targets transcripts by base pairing between one of the siRNA strands and the endogenous mRNA (Hammond, S. M., Bernstein, E., Beach, D., and Hannon, G. J. (2000) Nature 404: 293-96). A nuclease activity associated with the RISC complex then cleaves the mRNA-siRNA duplex thus targeting the cognate mRNA for destruction.

In mammalian cells the use of dsRNA to control gene expression has been hampered by the presence of a unique global response mechanism. Mammalian cells exposed to dsRNA longer than 30 base pairs in length trigger a response mechanism involving activation of two key enzymes, dsRNA-activated protein kinase (PKR) and 2′5′ oligoadenylate polymerase/RnaseL (Kumar, M., and Carmichael, G. G. (1998) Microbiol Mol Biol Rev 62(4), 1415-34). The activation of these enzymes leads to a cessation of protein synthesis and eventually cell death via apoptosis. It was thus anticipated that the introduction of long dsRNA would activate this global response system. However, studies have shown that in both mouse pre-implantation embryos (Svoboda, P., Stein, P., Hayashi, H., and Schultz, R. M. (2000) Development 127(19), 4147-4156; Wianny, F., and Zernicka-Goetz, M. (2000) Nat Cell Biol 2(2), 70-5) and undifferentiated embryonic stem cells and embryonic carcinoma cells (Yang, S., Tutton, S., Pierce, E., and Yoon, K. (2001) Mol Cell Biol 21(22), 7807-16; Billy, E., Brondani, V., Mang, H., Muller, U. and Filipowicz, W. (2001) Proc. Natl Acad Sci 98, 14428-14483; Paddison, P., Caudy, A. A., and Hannon, G. J. (2002) Proc. Natl Acad. Sci. 99, 1443-1448), the use of in vitro generated long dsRNA was able to mediate specific gene silencing. The primary reason for these observations was that these cell systems lack the generalised responses to dsRNA. These results were encouraging but placed particular limitations on the utility of this approach in differentiated mammalian cells.

Following on from observations that the products of the Dicer enzyme could mediate RNAi in Drosophila embryo extracts, it was then demonstrated that chemically synthesised 21 bp siRNAs could be used in a wide range of human and mouse cell lines to induce gene silencing (Elbashir, S. M., Harborth, J., Lendeckel, W., Yalcin, A., Weber, K., and Tuschl, T. (2001) Nature 411(6836), 494-8; Caplen, N. J., Parrish, S., Imani, F., Fire, A., and Morgan, R. A. (2001) Proc. Natl. Acad. Sci. 98, 9742-9747). This approach for transiently controlling the expression of a wide range of different target genes has been demonstrated and is becoming the method of choice for determining gene function in mammalian cells (Hsu, J. Y., Reimann, J. D. R., Sorensen, C. S., Lucas, J., and Jackson, P. K. (2002) Nature Cell Biol. 4, 358-366; Thompson, B., Tonwsley, F., Rosin-Arbesfeld, R., Muisi, H., and Bienz, M. (2002) Nature Cell Biol. 4, 367-373). One of the limitations associated with these synthetic dsRNA strategies is the transient nature of the suppressive effect induced by the dsRNA.

More recently, it has been shown that mammalian cells contain a very large group of small RNAs called microRNAs which are postulated to be transcribed as hairpin RNA precursors that are processed by Dicer to produce the mature 21 base forms (Lagos-Quintana, M., Rauhut, R., Lendeckel, W., and Tuschl, T. (2001) Science 294, 853-858; Lau, N. C., Lim, L. P., Weinstein, E. G., and Bartel, D. P. (2001) Science 294, 858-862; Lee, R. C. and Ambros, V. (2001) Science 294, 862-864). Several groups have exploited this naturally occurring biological mechanism to show that short hairpin RNAs (shRNAs) can induce specific gene silencing in mammalian cells (Paddison, P. J., Caudy, A. A., Bernstein, E., Hannon, G. J., and Conklin, D. S. (2002) Genes & Dev 16, 948-958; Brummelkamp, T. R., Bernards, R., and Agami, R. (2002) Science 296, 550-553; Sui, G., Soohoo, C., Affar, E., Gay, F., Shi, Y., Forrester, W. C., and Shi, Y. (2002) Proc Natl Acad Sci 99, 5515-20; Yu, J., DeRuiter, S. L., and Turner, D. L. (2002) Proc Natl Acad Sci 99, 6047-52). Furthermore, expression cassettes have been developed using the endogenous U6 snRNA or H1 promoters to drive expression of sequence-specific shRNAs that can regulate gene expression both transiently and stably in mammalian cells via RNAi (Paddison, P. J., Caudy, A. A., Bernstein, E., Hannon, G. J., and Conklin, D. S. (2002) Genes & Dev 16, 948-958; Brummelkamp, T. R., Bernards, R., and Agami, R. (2002) Science 296:550-553). ShRNAs produced from these expression cassettes were processed by Dicer to 21 bp siRNAs which are believed to be the effectors of gene silencing. It is anticipated that these cassettes will be useful for reverse genetic approaches in mammalian cells and transgenic mice to better understand gene function, and also as therapeutics.

A major limitation with the state of the art for RNAi in mammalian cells is the lack of any strategy for using RNAi knockdowns in a forward genetic approach to identify new genes involved in cellular processes or different human diseases. At present, synthetic siRNAs or RNAi expression constructs are designed on a gene-by-gene basis limiting the utility of these strategies for both generating and screening genome-wide RNAi expression libraries. The present invention provides methods which enable the production of RNAi libraries.

### SUMMARY OF THE INVENTION

In a first aspect the present invention provides a method of producing a DNA molecule wherein mRNA transcribed from the DNA molecule forms hairpin RNA (hRNA), the method comprising:

(i) synthesizing a first DNA strand comprising in order a first sequence, a random sequence and a second sequence, wherein nucleotides at the 3′ end of the second sequence are complementary to nucleotides at the 5′ end of the second sequence such that the second sequence forms a stem loop;
(ii) synthesizing a complementary DNA strand extending from the stem loop using a DNA polymerase, the complementary DNA strand being complementary to the first sequence and the random sequence so as to form hairpin DNA;
(iii) denaturing the hairpin DNA to form a single DNA strand; and
(iv) adding a primer which hybridises to the complement of the first sequence and DNA polymerase to synthesize double stranded DNA.

In a second aspect the present invention provides a method of preparing an expression vector, expression of which produces double stranded RNA (dsRNA), the method comprising:

(i) synthesizing a first DNA strand comprising in order at least four consecutive adenosine nucleotides, a random sequence, at least four consecutive thymidine nucleotides and a primer binding site;
(ii) annealing a primer to the primer binding site and synthesizing a second DNA strand which is substantially complementary to the first DNA strand and forms double stranded DNA; and
(iii) cloning the double stranded DNA into an expression vector between two convergent promoters.

In a third aspect the present invention provides a method for determining a function of a gene, the method comprising:

(i) synthesizing a first DNA strand comprising in order a first sequence, a random sequence and a second sequence, wherein nucleotides at the 3′ end of the second sequence are complementary to nucleotides at the 5′ end of the second sequence such that the second sequence forms a stem loop;
(ii) synthesizing a complementary DNA strand extending from the stem loop using a DNA polymerase, the complementary DNA strand being complementary to the first region and the random sequence so as to form hairpin DNA;
(iii) denaturing the hairpin DNA to form a single DNA strand;
(iv) adding a primer which hybridises to the complement of the first sequence and DNA polymerase to synthesize double stranded DNA;
(v) cloning the double stranded DNA into an expression vector wherein the double stranded DNA is under the control of a promoter;
(vi) transfecting an effective amount of the expression vector into a cell under conditions permitting transcription of the double stranded DNA to produce a transfected cell; and
(vii) detecting one or more changes in the transfected cell relative to a control cell.

In a fourth aspect the present invention provides a method for determining a function of a gene, the method comprising:

(i) synthesizing a first DNA strand comprising in order at least four consecutive adenosine nucleotides, a random sequence, at least four consecutive thymidine nucleotides and a primer binding site;
(ii) annealing a primer to the primer binding site and synthesizing a second DNA strand which is substantially complementary to the first DNA strand and forms double stranded DNA;
(iii) cloning the double stranded DNA into an expression vector between two convergent promoters;
(iv) transfecting an effective amount of the expression vector into a cell under conditions favouring transcription of the double stranded DNA to produce a transfected cell; and
(v) detecting one or more changes in the transfected cell relative to a control cell.

In a fifth aspect, the present invention provides an expression vector for use in suppressing expression of a target gene, the vector comprising a pair of convergent promoters and a DNA molecule positioned therebetween, the DNA molecule comprising a target-specific sequence flanked by two directional transcription terminators, the target-specific sequence comprising a sequence of at least 14 nucleotides having at least 90% identity to a segment of the target gene.

In a sixth aspect the present invention provides a method for determining a function of a target gene, the method comprising:

(i) preparing an expression vector comprising a pair of convergent promoters and a DNA molecule positioned therebetween, the DNA molecule comprising a target-specific sequence flanked by two directional transcription terminators, the target-specific sequence comprising a sequence of at least 14 nucleotides having at least 90% identity to a segment of the target gene;
(ii) transfecting an effective amount of the expression vector into a cell to produce a transfected cell; and
(iii) detecting one or more phenotypic changes in the transfected cell relative to a control cell.

In a seventh aspect the present invention provides a method of producing a library of DNA molecules wherein mRNA transcribed from the DNA molecules forms hairpin RNA (hRNA) molecules, the method comprising:

(i) preparing a library of double stranded DNA fragments;
(ii) ligating hairpin DNA to the DNA fragments from step (i);
(iii) ligating a double stranded DNA adaptor to the DNA from step (ii), wherein the DNA adaptor includes a primer binding site;
(iv) denaturing the DNA from step (iii) to form a library of single DNA strands; and
(v) adding a primer which hybridises to the primer binding site and DNA polymerase to synthesize double stranded DNA thereby producing a library of double stranded DNA molecules.

In an eighth aspect the present invention provides a method of preparing a library of expression vectors, expression of which produces double stranded RNA (dsRNA) molecules, the method comprising:

(i) preparing a library of double stranded DNA fragments;
(ii) ligating a double stranded DNA adaptor to each end of the DNA fragments from step (i), wherein the sequence of the DNA adaptor comprises at least four consecutive adenosine nucleotides at the 3′ end; and
(iii) cloning the double stranded DNA from step (ii) into an expression vector between two convergent promoters.

In a ninth aspect the present invention provides a method of producing a library of DNA molecules wherein mRNA transcribed from the DNA molecules forms hairpin RNA (hRNA) molecules, the method comprising:

(i) preparing a pool of mRNA;
(ii) adding an enzyme to the pool of mRNA, wherein the enzyme reverse transcribes the mRNA to form cDNA and degrades the mRNA;
(iii) allowing the cDNA from step (ii) to form a hairpin loop;
(iv) synthesising a second strand using the hairpin loop as a priming point for reverse transcriptase;
(v) denaturing the DNA from step (iv) to form single stranded DNA; and
(vi) adding DNA polymerase to synthesize double stranded DNA thereby producing a library of double stranded DNA molecules.

In a further aspect the present invention provides a method of inhibiting expression of a target gene in a cell, the method comprising introducing into the cell an expression vector according to the fifth aspect of the present invention.

### BRIEF DESCRIPTION OF THE FIGURES

FIG. 1. Enzymatic generation of DNA insert encoding a p53-specific shRNA. The six steps involved in the generation of a double-stranded DNA insert encoding a shRNA specific for human p53. Abbreviations: sal1RE=SalI restriction enzyme site; U=deoxyribouridine; p53=19 bases specific to sense strand of p53 mRNA; stem loop=21 bases constituting stem loop structure.

FIG. 2. Enzymatic generation of DNA insert encoding a random shRNA. The six steps involved in the generation of a double-stranded DNA insert encoding a shRNA for any random sequence. The abbreviations are the same as indicated in FIG. 1, with the following exceptions: N19=random 19 bases; As19=antisense of the random N19 sequence; Nc19=complementary DNA strand to N19; Asc19=complementary DNA strand to As19.

FIG. 3. Suppression of dEGFP-mediated cell fluorescence using a EGFP-specific shRNA expression plasmid. A. Flow cytometry analysis of HEK 293 cells (containing a stably integrated dEGFP target gene) transiently transfected with pTZ(U6+1) vector alone (purple) or pTZ(U6+1)GFP (green overlay). B. Quantitation of the FACs analysis represented in A. Each sample was transfected in triplicate.

FIG. 4. Construction of random shRNA expression library in a modified pLXSN retroviral vector. A. The 45 bp stuffer fragment containing a unique SwaI site was introduced between the SalI and XbaI sites downstream of the U6 promoter. B. Cloning site in pLXSNU6Swa. C. Generation of random shRNA expression vector using pLXSNU6Swa.

FIG. 5. Enzymatic generation of DNA insert encoding complementary sense and antisense RNAs specific for p53. The four steps involved in generating the DNA insert encoding complementary sense and antisense RNAs specific for human p53.

FIG. 6. Reduction in dEGFP-mediated cell fluorescence in cells transiently transfected with a retroviral expression vector encoding EGFP siRNA. A. Structure of the retroviral vector pLXSNU6/H1GFP encoding EGFP-specific siRNA. B. Suppression of dEGFP-mediated cell fluorescence in HEK 293 cells (containing a stably integrated dEGFP transgene) after infection with pLXSNU6/H1GFP.

FIG. 7. Reduction in p53 protein levels in HCT116 colon carcinoma cells infected with a retroviral expression vector encoding p53 siRNA. Structure of the pLXSNU6/H1p53 retroviral siRNA expression vector.

FIG. 8. Construction of a genome-wide siRNA retroviral expression library. A. Overview of the four steps used to generate random inserts and construct a random siRNA expression library. B. Schematic of the structure of the random siRNA expression vector system. C. Distribution of library inserts in the human genome.

FIG. 9. Strategy for generating intracellular siRNAs and effect of the expressed siRNAs on transgene expression. A. The convergent U6 expression cassette encodes sense and antisense RNAs that terminate at directional termination sequences. The complementary RNAs anneal and undergo further Dicer-dependent processing to produce functional siRNAs. B. A U6 convergent expression vector containing an EGFP-specific insert (DualU6GFP) reduces dEGFP-mediated cell fluorescence.

FIG. 10. Suppression of dEGFP transgene expression using a stably integrated convergent transcription vector. HEK 293 cells were cotransfected with either the pDualU6 vector or pDualU6GFP and the pREP7 plasmid in a 10:1 molar ratio, and cells selected for resistance to hygromycin. Following selection, cells were examined for level of dEGFP-mediated cell fluorescence.

FIG. 11. Suppression of target gene expression by the DualU6GFP vector requires the co-expression of both sense and antisense RNAs.

FIG. 12. The DualU6GFP expression vector reduces dEGFP target gene expression in a Dicer-dependent manner.

FIG. 13. 5-FU-induced apoptosis in HCT116 cells containing pLXSNU6/H1p53. A. Decrease in subG1 population in cells expressing p53 siRNA. B. Cells expressing p53 siRNA display resistance to 5-FU-induced activation of caspase. C. Cells expressing p53 siRNA show increased cell viability following exposure to 5-FU.

FIG. 14. Overview of the 5-FU genetic selection of spiked siRNA expression libraries.

FIG. 15. Retroviral expression vectors for genome-wide RNAi libraries. A. pLXSNU6/H1. B. pLXSNU6/H1LTR. C. pQCXINU6/H1SIN.

FIG. 16 Method for constructing genome-specific shRNA and siRNA libraries.

FIG. 17. Schematic overview of the method for constructing shRNA and siRNA libraries specific for an expressed RNA population.

FIG. 18 Identification of HIV-specific shRNA or siRNA using genetic selections.

### DETAILED DESCRIPTION OF THE INVENTION

The present invention relates to methods which enable production of a library of DNA sequences encoding shRNA or siRNAs that are capable of recognising all target mRNA sites to identify, isolate and characterise unknown and known genes that contribute to a specific cellular phenotype or are modified by specific stimuli. These expression libraries are designed to suppress the expression of a target gene and based on the sequence of the encoded shRNA or siRNA identify the target gene responsible for the change in cellular phenotype. This method requires the construction of random shRNA and siRNA expression libraries that contain inserts encoding RNA sequences that form double-stranded RNA via intramolecular or intermolecular hybridisation in vivo, respectively.

The present invention also provides a convergent promoter system capable of producing sense and antisense RNAs that mediate gene silencing in mammalian cells through the RNAi pathway. This system can be used to inhibit transgene and endogenous gene expression. The use of dsRNA as a mediator has distinct advantages over hammerhead and hairpin ribozymes including the presence of a natural cellular protein complex (termed RISC) that binds the expressed dsRNA and mediates interaction with the target mRNA and cleavage of the target mRNA.

In a first aspect the present invention provides a method of producing a DNA molecule wherein mRNA transcribed from the DNA molecule forms hairpin RNA (hRNA), the method comprising:

(i) synthesizing a first DNA strand comprising in order a first sequence, a random sequence and a second sequence, wherein nucleotides at the 3′ end of the second sequence are complementary to nucleotides at the 5′ end of the second sequence such that the second sequence forms a stem loop;
(ii) synthesizing a complementary DNA strand extending from the stem loop using a DNA polymerase, the complementary DNA strand being complementary to the first sequence and the random sequence so as to form hairpin DNA;
(iii) denaturing the hairpin DNA to form a single DNA strand; and
(iv) adding a primer which hybridises to the complement of the first sequence and DNA polymerase to synthesize double stranded DNA.

In a preferred embodiment a deoxyuracil nucleotide is included in the first sequence and prior to addition of the primer the single DNA strand is depurinated, preferably with uracil nucleotide glycosylase, and β-eliminated.

In a preferred embodiment the double stranded DNA is cloned into an expression vector. More preferably the double stranded DNA is cloned into an expression vector wherein the double stranded DNA is under the control of a promoter.

In a preferred embodiment the first DNA strand includes a restriction enzyme site. Delivery and transcription of the expression vectors of the present invention in a host cell provides a hRNA, in particular, short hairpin RNA (shRNA) specific for a target mRNA having complementarity with the double-stranded RNA region. The shRNAs of the invention have been shown to be effective modifiers of gene expression. Preferably the random sequence is about 19 to about 30 base pairs in length. More preferably the random sequence is from 19 to 25 base pairs in length. Most preferably the random sequence is 19 base pairs in length.

As used herein, the term “complementary” is used in reference to “polynucleotides” and oligonucleotides” (which are interchangeable terms that refer to a sequence of nucleotides) related by the base pairing rules. For example, the sequence 5′-CTGAG-3′ is complementary to the sequence 5′-CTCAG-3′. Complementarity can be partial or total. Partial complementarity is where one or more nucleic acid bases is not matched according to the base pairing rules. Total or complete complementarity is where each and every nucleic acid base is matched with another base according to base pairing rules. The degree of complementarity between nucleic acid strands has significant effects on the efficiency and strength of hybridisation between nucleic acid strands.

The term “loop” refers to an unpaired secondary structure in a nucleic acid sequence in which a single-stranded nucleic acid sequence is flanked by nucleic acid sequences which are capable of pairing with each other to form a “stem” structure. The term “unpaired” when made in reference to nucleic acid refers to a secondary structure in an nucleic acid sequence in which nucleic acid is single-stranded and is flanked by nucleic acid sequences which are incapable of pairing with each other, but which are capable of pairing with other sequences. Loop structures of any length and any sequence are contemplated to be within the scope of this invention. Computer programs for the prediction of RNA secondary structure formation are known in the art and include, for example, “RNAFOLD” described in Hofacker et al. (1994) Monatshefte F. Chemie 125:167-188; McCaskill (1990) Biopolymers 29:1105-1119 and “DNASIS” (Hitachi).

The term “expression vector” as used herein refers to a recombinant DNA molecule containing a desired coding sequence and appropriate nucleic acid sequences necessary for the expression of the operably linked coding sequence in a host organism. Nucleic acid sequences necessary for expression in eukaryotic cells usually include a promoter and termination and polyadenylation signals. In a preferred embodiment the expression vector also incorporates stabilisation elements into the expressed RNA to increase the stability of the RNA. As used herein, the term “vector” is used in reference to nucleic acid molecules that transfer DNA segment(s) from one cell to another. Vector includes plasmids, viruses, retrotransposons and cosmids.

Preferably the double stranded DNA is cloned into an expression vector suitable for expression in a mammalian cell. Methods which are well known to those skilled in the art can be used to construct expression vectors containing a sequence which encodes the RNA expression library. These methods include in vitro recombinant DNA techniques, synthetic techniques and in vivo recombination or genetic recombination. Such techniques are described in Sambrook et al (1989) Molecular Cloning, A laboratory Manual, Cold Spring Harbor Press, Plainview N.Y. and Ausbel F M et al (1989) Current Protocols in Molecular Biology, John Wiley & Sons, New York N.Y.

As used herein, the term “promoter” refers to a single promoter sequence as well as to a plurality (i.e., one or more) of promoter sequences which are operably linked to each other and to at least one DNA sequence of interest. Promoters consist of short arrays of DNA sequences that interact specifically with cellular proteins involved in transcription (Maniatis T. et al., Science 236:1237 (1987). Promoter elements have been isolated from a variety of eukaryotic sources including genes in plant, yeast, insect and mammalian cells and viruses.

The selection of a particular promoter depends on what cell type is to be used to express the DNA sequence of interest. The promoter also optionally includes distal enhancer or repressor elements which can be located as much as several thousand base pairs from the site of transcription. The promoter may be constitutive, such as a promoter active under most environmental conditions or stages of development or the promoter may be inducible, and respond to, for example, an extracellular stimulus.

Efficient expression of recombinant DNA sequences in eukaryotic cells requires expression of signals directing the efficient termination and polyadenylation of the resulting transcript. Transcription termination signals are generally found downstream of the polyadenylation signal and are generally a few hundred nucleotides in length.

In a preferred embodiment the double stranded DNA is cloned into an expression vector under the control of a U6 snRNA, H1 or T7 promoter. More preferably the double stranded DNA is cloned into an expression vector under the control of a U6 snRNA promoter. Synthesis of the second DNA strand may be achieved using second strand synthesis techniques well known to those of skill in the art for synthesizing a second strand of DNA from a first strand of DNA, for example utilizing a DNA polymerase such as AmpliTaq DNA polymerase (Perkin Elmer). Suitable techniques for second strand synthesis may be as set out in Sambrook et al (1989) Molecular Cloning, A laboratory Manual, Cold Spring Harbor Press, Plainview N.Y. and Ausbel F M et al (1989) Current Protocols in Molecular Biology, John Wiley & Sons, New York N.Y.

In a second aspect the present invention provides a method of preparing an expression vector, expression of which produces double stranded RNA (dsRNA), the method comprising:

(i) synthesizing a first DNA strand comprising in order at least four consecutive adenosine nucleotides, a random sequence, at least four consecutive thymidine nucleotides and a primer binding site;
(ii) annealing a primer to the primer binding site and synthesizing a second DNA strand which is substantially complementary to the first DNA strand and forms double stranded DNA; and
(iii) cloning the double stranded DNA into an expression vector between two convergent promoters.

Transcription from the convergent promoters of two strands of the resident inserts results in the production of two small complementary RNAs that are capable of hybridising to form an siRNA with two to four base overhangs at their 3′ ends.

The expression vector produced according to the methods of the invention are useful in identifying the function of a gene or sequence of interest in an organism. Preferably the random sequence is about 19 to about 30 base pairs in length. More preferably the random sequence is from 19 to 25 base pairs in length. Most preferably the random sequence is 19 base pairs in length.

In a preferred embodiment the double stranded DNA is cloned into an expression vector between two convergent U6 snRNA, H1 or T7 promoters. More preferably the double stranded DNA is cloned into an expression vector between two convergent U6 snRNA promoters.

The random sequence of the first or second aspect of the present invention may be produced in a number of ways including synthetic generation by random insertion of nucleotides during synthesis, by use of an EST library or by random digestion of the genome of the organism of interest. Production of a library by random digestion of a genome may be of particular interest in analysing gene function in viral and other pathogens. Random digestion of a genome may be achieved by techniques known to those of skill in the art, such as DNAse I digestion. Synthetic sequences may be generated chemically according to known methods such as the solid phase phosphoramidite triester method described by Beaucage and Caruthers (1981) Tetrahedron Letts. 22(20):1859-1862, e.g. using an automated synthesiser as described in Needham-VanDevanter et al (1984) Nucleic Acids Res., 12:6159-6168. Purification of the molecule, where necessary, is typically performed by either gel electrophoresis or by anion-exchange HPLC as described in Pearson and Regnier (1983) J. Chrom. 255:137-149. The sequence can be verified using the chemical degradation method of Maxam and Gilbert (1980) in Grossman and Moldave (eds.) Academic Press, New York, Methods in Enzymology 65:499-560.

In a preferred embodiment, the expression vectors prepared according to the methods of the first or second aspect are used to transfect a host cell.

In a third aspect the present invention provides a method for determining a function of a gene, the method comprising:

(i) synthesizing a first DNA strand comprising in order a first sequence, a random sequence and a second sequence, wherein nucleotides at the 3′ end of the second sequence are complementary to nucleotides at the 5′ end of the second sequence such that the second sequence forms a stem loop;
(ii) synthesizing a complementary DNA strand extending from the stem loop using a DNA polymerase, the complementary DNA strand being complementary to the first region and the random sequence so as to form hairpin DNA;
(iii) denaturing the hairpin DNA to form a single DNA strand;
(iv) adding a primer which hybridises to the complement of the first sequence and DNA polymerase to synthesize double stranded DNA;
(v) cloning the double stranded DNA into an expression vector wherein the double stranded DNA is under the control of a promoter;
(vi) transfecting an effective amount of the expression vector into a cell under conditions permitting transcription of the double stranded DNA to produce a transfected cell; and
(vii) detecting one or more changes in the transfected cell relative to a control cell.

In a fourth aspect the present invention provides a method for determining a function of a gene, the method comprising:

(i) synthesizing a first DNA strand comprising in order at least four consecutive adenosine nucleotides, a random sequence, at least four consecutive thymidine nucleotides and a primer binding site;
(ii) annealing a primer to the primer binding site and synthesizing a second DNA strand which is substantially complementary to the first DNA strand and forms double stranded DNA;
(iii) cloning the double stranded DNA into an expression vector between two convergent promoters;
(iv) transfecting an effective amount of the expression vector into a cell under conditions favouring transcription of the double stranded DNA to produce a transfected cell; and
(v) detecting one or more changes in the transfected cell relative to a control cell.

In a fifth aspect the present invention provides an expression vector for use in suppressing expression of a target gene, the vector comprising a pair of convergent promoters and a DNA molecule positioned therebetween, the DNA molecule comprising a target-specific sequence flanked by two directional transcription terminators, the target-specific sequence comprising a sequence of at least 14 nucleotides having at least 90% identity to a segment of the target gene.

Delivery and transcription of the expression vectors of the present invention in a host cell provides an siRNA or hRNA specific for a target mRNA having complementarity with the target-specific sequence. The siRNAs of the invention have been shown to be effective modifiers of gene expression.

Preferably the target-specific sequence is at least 19 base pairs in length. More preferably the target-specific sequence is 19 to about 30 base pairs in length. More preferably the target-specific sequence is from 19 to 25 base pairs in length. Most preferably the target-specific sequence is 19 base pairs in length.

The target gene may be any gene of interest, the manipulation of which may be deemed desirable for any reason, by one of ordinary skill in the art.

In a preferred embodiment the target-specific sequence has at least 95% identity, and more preferably is identical, to a segment of the target gene.

In a preferred embodiment the expression vector is a retroviral expression vector.

In a preferred embodiment the expression vector encodes a selectable marker, for example an antibiotic resistance gene, for selection of cells transfected with the expression vector. More preferably the expression vector encodes the G418 selection marker.

Methods which are well known to those skilled in the art can be used to construct expression vectors of the present invention. These methods include in vitro recombinant DNA techniques, synthetic techniques and in vivo recombination or genetic recombination. Such techniques are described in Sambrook et al (1989) Molecular Cloning, A laboratory Manual, Cold Spring Harbor Press, Plainview N.Y. and Ausbel F M et al (1989) Current Protocols in Molecular Biology, John Wiley & Sons, New York N.Y.

Transcription from the convergent promoters of two strands of the resident inserts results in the production of two small complementary RNAs that are capable of hybridising to form an siRNA with two to four base overhangs at their 3′ ends.

In a preferred embodiment the convergent promoters are U6 snRNA, H1 or T7 promoters. More preferably the convergent promoters are U6 snRNA promoters.

The expression vector produced according to the methods of the invention are useful in identifying the function of a gene or sequence of interest in an organism.

In a sixth aspect the present invention provides a method for determining a function of a target gene, the method comprising:

(i) preparing an expression vector comprising a pair of convergent promoters and a DNA molecule positioned therebetween, the DNA molecule comprising a target-specific sequence flanked by two directional transcription terminators, the target-specific sequence comprising a sequence of at least 14 nucleotides having at least 90% identity to a segment of the target gene;
(ii) transfecting an effective amount of the expression vector into a cell to produce a transfected cell; and
(iii) detecting one or more phenotypic changes in the transfected cell relative to a control cell.

The present invention provides methods for the identification of one or more functions of a nucleotide sequence in an organism. The methods of the invention selectively reduce, diminish or destroy the RNA encoded by the targeted coding sequence in order to render the RNA non-functional while the targeted gene in the host remains intact. These methods therefore employ a “knockdown” strategy to determine gene function instead of the traditional “knockout” methods. The invention is useful for the rapid identification of, for example, disease related genes which may be targeted for the treatment or prevention of disease. The methods of the present invention also have utility in identifying viral or pathogen-derived genes that play a major role in the susceptibility of cells to infection by viruses or pathogens.

In a preferred embodiment the expression vector is a retroviral expression vector.

In a preferred embodiment the transfected cell is recovered and the double stranded DNA insert recovered or amplified by, for example, using the polymerase chain reaction, re-cloned and subjected to additional enrichment steps.

In a further preferred embodiment the enriched insert is sequenced and used to identify potential target genes by, for example, homology searching, or utilised to capture the target mRNA.

In a preferred embodiment the expression vector encodes a selectable marker, for example an antibiotic resistance gene, for selection of cells transfected with the expression vector. More preferably the expression vector encodes the G418 selection marker.

The term “transfection” as used herein refers to the introduction of a transgene, for example a vector, into a cell. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection or biolistics (i.e., particle bombardment). Transfection may be transient or stable transfection. The term “stable transfection” or “stably transfected” refers to the introduction and integration of a transgene into the genome of a transfected cell. The term “transient transfection” or “transiently transfected” refers to the introduction of one or more transgenes into a transfected cell in the absence of integration of the transgene into the genome of the host cell.

The term “gene of interest” refers to any gene, the manipulation of which may be deemed desirable for any reason, by one of ordinary skill in the art.

In a preferred embodiment the methods of the present invention for determining the function of a genomic DNA sequence, a shRNA or siRNA sequence is introduced into a cell in order to reduce the amount of RNA expressed by that genomic sequence.

It is desirable to express a sufficient amount of shRNA or siRNA such that substantially all the substrate RNA is cleaved. Such substantial abrogation of substrate RNA expression would facilitate the observation of the effect of depletion of gene function in the organism wherein the shRNA or siRNA is expressed. While desirable, complete elimination of the substrate RNA is not required by the methods of the invention.

A “control” cell as used herein includes a cell that is untransfected, has been mock transfected, or has been transfected with an “empty vector” such as an expression vector without the double stranded DNA insert.

Host cells, such as eukaryotic cells, harbouring the expression vectors described above also are provided by this invention. Suitable host cells include, but are not limited to, bacterial cells, rat cells, mouse cells and human cells.

The methods of the invention are useful for determining the function of a gene or DNA sequence of interest in an organism by forward genetic approaches including observing the effects of reducing expression of the gene or DNA sequence in the organism or of a homologous gene or DNA sequence in another organism. For example, data presented herein demonstrates that the function of the p53 or EGFP gene in HCT116 colon cancer cells or HEK 293 embryonic kidney cells respectively may be determined by siRNA or shRNA mediated cleavage of transcripts.

The types of genetic selections that can be used in a forward genetic approach with a genome-wide RNAi library includes overcoming cell growth arrest by, for example, bypassing p53-mediated growth arrest and apoptosis; identifying new targets involved in chemotherapeutic drug resistance such as overcoming 5-FU-induced growth arrest, apoptosis and senescence; blocking activated signalling pathways, for example, identifying novel positive and negative regulators of signalling pathways implicated in cancer, such as the TGFβ and Wnt pathways; elucidating resistance to viral and pathogen infection including genetic screens for genes that confer resistance to HIV infection or that interfere with the productive or latent phases of the viral life cycle or genetic screens for genes that interfere with the lifecycle of an intracellular parasite such as plasmodium; synthetic lethality screens to identify gene products whose inactivation leads to cell death, particularly in tumor cells deficient for either the p53 or p16/Rb tumor suppression pathways; identifying genes involved in metastasis, for example using in vivo assays; identifying optimal siRNAs against specific target(s); detecting genes regulating specific promoters; detecting cell cycle regulatory genes, for example using soft agar assays (for anchorage dependent growth) and minimal medium (for growth factor-independent growth), both of which are widely used indicators of cellular transformation in cell culture; identifying unknown genes responsible for tumorigenesis such as using bromo-deoxyuridine, a nucleoside analog that is toxic to cells undergoing active division.

As will be appreciated by those skilled in this field the present invention allows the production of libraries of constructs the expression of which result in siRNA or hRNA. Accordingly, in a seventh aspect the present invention provides a method of producing a library of DNA molecules wherein mRNA transcribed from the DNA molecules forms hairpin RNA (hRNA) molecules, the method comprising:

(i) preparing a library of double stranded DNA fragments;
(ii) ligating hairpin DNA to the DNA fragments from step (i);
(iii) ligating a double stranded DNA adaptor to the DNA from step (ii), wherein the DNA adaptor includes a primer binding site;
(iv) denaturing the DNA from step (iii) to form a library of single DNA strands; and
(v) adding a primer which hybridises to the primer binding site and DNA polymerase to synthesize double stranded DNA thereby producing a library of double stranded DNA molecules.

In an eighth aspect the present invention provides a method of preparing a library of expression vectors, expression of which produces double stranded RNA (dsRNA) molecules, the method comprising:

(i) preparing a library of double stranded DNA fragments;
(ii) ligating a double stranded DNA adaptor to each end of the DNA fragments from step (i), wherein the sequence of the DNA adaptor comprises at least four consecutive adenosine nucleotides at the 3′ end; and
(iii) cloning the double stranded DNA from step (ii) into an expression vector between two convergent promoters.

In a preferred embodiment the library of double stranded DNA fragments is prepared by digestion of DNA. The DNA that is digested is preferably a gene, a genome or cDNA library. The digestion may be carried out using a range of enzymes well known in the field, however, it is preferred that the digestion is with DNAseI.

The resulting double stranded DNA is preferably cloned into an expression vector under the control of a promoter selected from the group consisting of U6 snRNA, H1 and T7, preferably a U6 snRNA promoter.

In a ninth aspect the present invention provides a method of producing a library of DNA molecules wherein mRNA transcribed from the DNA molecules forms hairpin RNA (hRNA) molecules, the method comprising:

(i) preparing a pool of mRNA;
(ii) adding an enzyme to the pool of mRNA, wherein the enzyme reverse transcribes the mRNA to form cDNA and degrades the mRNA;
(iii) allowing the cDNA from step (ii) to form a hairpin loop;
(iv) synthesising a second strand using the hairpin loop as a priming point for reverse transcriptase;
(v) denaturing the DNA from step (iv) to form single stranded DNA; and
(vi) adding DNA polymerase to synthesize double stranded DNA thereby producing a library of double stranded DNA molecules.

In a preferred embodiment the enzyme in step (ii) is AMV reverse transcriptase.

It is further preferred that the double stranded DNA molecules are cloned into expression vectors under the control of a promoter selected from the group consisting of U6 snRNA, H1 and T7, preferably under the control of a U6 snRNA promoter.

The ability to express siRNAs that act through the RNAi pathway allows for regulation of expression of genes and therapeutic applications to alleviate disease states resulting from expression of these genes.

Accordingly, in a further aspect the present invention provides a method of inhibiting expression of a target gene in a cell, the method comprising providing the cell with an expression vector according to the fifth aspect of the invention.

The target gene may be a gene derived from a cell of the organism, a transgene, or a gene of a pathogen present in a cell of the organism, or remaining in the cell after infection by the pathogen.

The cell maybe an animal or plant cell and may be isolated or form part of a complete organism.

When used with an organism the expression vector of the fifth aspect may be provided to the organism by direct introduction, such as direct injection, or introduced by other means known to those of skill in the art including oral introduction or topical application. The expression vector may be introduced into a germ line or somatic cell, stem cell or other multipotent cell derived from the organism and re-introduced into the organism.

The present invention may be used for treatment or prevention of a disease state resulting from expression of the target gene. Disease states include, but are not limited to, autoimmune diseases, inherited diseases, cancer, infection by a pathogen or overexpression of the target gene. Treatment would include prevention or amelioration of any symptom or clinical indication associated with the disease.

Target genes according to the present invention include, but are not limited to, genes involved in chemotherapeutic drug resistance, apoptosis and senescence; genes implicated in cancer including genes involved in metastasis and genes responsible for tumorigenesis. The present invention also includes pharmaceutical compositions and formulations, which comprise at least one expression vector of the invention. The pharmaceutical compositions of the present invention may be administered in a number of ways depending upon whether local or systemic treatment is desired and upon the area to be treated. The administration can be topical, pulmonary, oral or parenteral.

Pharmaceutical compositions and formulations for topical administration may include transdermal patches, ointments, lotions, creams, gels, drops, suppositories, sprays, liquids and powders. Conventional pharmaceutical carriers, aqueous, powders or oily bases, thickeners and the like may be necessary or desirable.

Composition and formulations for oral administration include powders or granules, suspensions or solutions in water or non-aqueous media, capsules satchels or tablets. The expression vectors of the present invention can additionally be used to increase the susceptibility of tumour cells to anti-tumour therapies such as chemotherapy and radiation therapy.

Accordingly in certain embodiments of this invention there are provided liposomes and other compositions containing (a) one or more expression vectors of the invention and (b) one or more chemotherapeutic agents which function by a non-hybridisation mechanism. Examples of such chemotherapeutic agents include, but are not limited to, anticancer drugs such as taxol, daunorubicin, dacitinomycin, doxorubicin, bleomycin, mitomycin, nitrogen mustard, chlorambucil, melphalan, cyclophosphamide, 6-mercaptopurine, 6-thioguanine, cytarabine, 5-flurouracil, floxuridine, methotrexate, colchicine, vincristine, vinlastin, etoposide, cisplatin. See, generally, The Merck Manual of Diagnosis and Therapy, 15th Ed., Berkow et al eds., 1987, Rahway, N.J., pp 1206-1228.

The formulation of the therapeutic compositions and their subsequent administration is believed to be within the skill of those in the art. Dosing is dependent on severity and responsiveness of the disease state to be treated, with the course of treatment lasting from several days to several months, or until a cure is effected or diminution of the disease state is achieved. Optimal dosing schedules can be determined from measurements of drug accumulation in the body of the patient. Persons of ordinary skill can easily determine optimum dosages, dosing methodologies and repetition rates. In general, dosage is from 0.01 μg to 100 g per kg of body weight and may be given daily, weekly, monthly or yearly. Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application. In order that the nature of the present invention may be more clearly understood preferred forms thereof will now be described with reference to the following non-limiting Examples.

### Example 1

### Random ShRNA Library

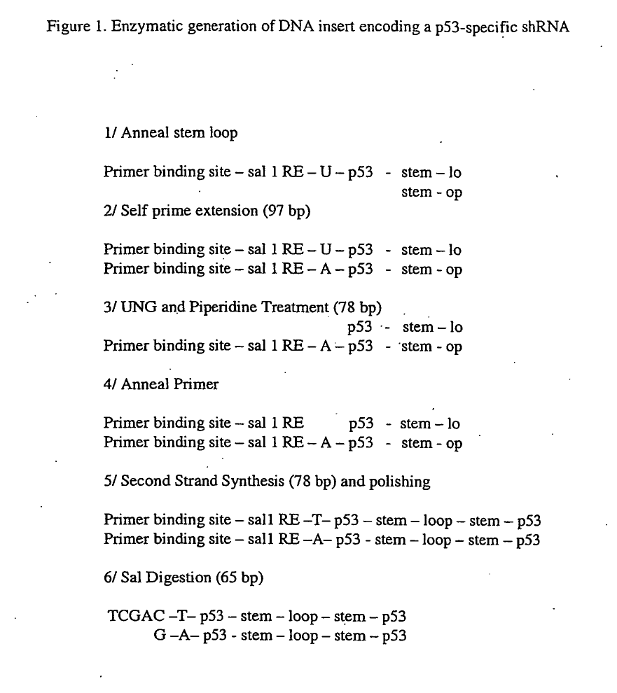

The following describes the methodology developed for generating random shRNA inserts and testing gene-expressed shRNAs for suppressing specific gene expression. In order to demonstrate the enzymatic protocol to generate a DNA insert encoding a shRNA, we used the p53 gene as a target (FIG. 1). The starting material for these reactions was the following oligonucleotide:

5′-TGTGGTGATTCGTCGACUGACTCCAGTGGTAATCTACGTCGAGTCTCTTGAACTCGAC-3′. (SEQ ID NO:1)

This template is composed of a primer binding site (TGTGGTGATTCGTCGAC) (SEQ ID NO:2), encompassing a SalI restriction enzyme site (underlined), a single deoxyribouridine base (bold), 19 nucleotides specific to human p53 (GACTCCAGTGGTAATCTAC) (SEQ ID NO:3) and a 21 base sequence capable of forming a stem loop (GTCGAGTCTCTTGAACTCGAC) (SEQ ID NO:4). The structure formed by the latter sequence is composed of six complementary bases flanking a loop sequence. The initial step in this methodology is the self-annealing of the internal stem loop structure (Step 1). This involves incubation of the oligonucleotide at 75° C. for 5 minutes followed by 37° C. for 20 minutes and 4° C. overnight. Following the annealing reaction, T4 DNA polymerase was used to extend the complementary antisense strand (Step 2). The hairpin structure formed was then subjected to depurination of the deoxyribouridine (U) by uracil DNA glycosylase, which was then β-eliminated by piperidine treatment, resulting in the loss of the fragment 5′ of the deoxyribouridine base (Step 3). Removal of this sequence exposes the primer-binding site. Following annealing of the primer sequence (TGTGGTGATTCGTCGAC) (SEQ ID NO:2), second strand synthesis was performed using a DNA polymerase capable of strand displacement (for example, Bst DNA polymerase) (Steps 4 and 5). The double-stranded DNA was digested with SalI and ligated to the appropriate vector (see below) (Step 6).

To generate a library of random shRNA-encoding inserts for use in constructing a genome-wide RNAi expression library, we synthesised the following oligonucleotide:

### 5′-TGTGGTGATTCGTCGACUNNNNNNNNNNNNNNNNNNNGTCGAGTCTCTTGAACTCGAC-3′ (SEQ ID NO:5)

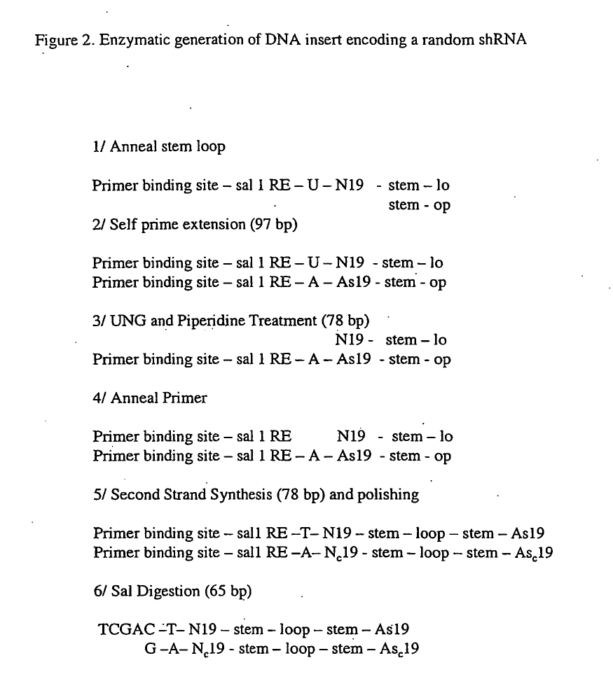

A total of 1 μmole of this sequence was synthesised using a special hand mix to ensure equimolar ratios of A, T, C and G (Integrated DNA Technologies, USA). This sequence was subjected to the enzymatic steps indicated in FIG. 1 to produce double-stranded DNA inserts each encoding a unique shRNA (FIG. 2). The DNA inserts were digested with SalI and cloned into a suitable expression vector under control of a RNA polymerase II or III promoter (see below). In addition, these vectors contain the appropriate transcriptional terminator sequence.

To examine the suitability of the DNA inserts, encoding shRNAs, for suppressing the expression of a specific gene we chose dEGFP as a target. To construct pTZ(U6+1)GFP, encoding a EGFP-specific shRNA, the two oligonucleotides 5′-TCGACCGGCAAGCTGACCCTGAAGTTCGCTTCAGGGTCAGCTTGCCGTTTTT-3′ (SEQ ID NO:6) and 5′-CTAGAAAAACGGCAAGCTGACCCTGAAGCGAACTTCAGGGTCAGCTTGCCCG-3′ (SEQ ID NO:7)

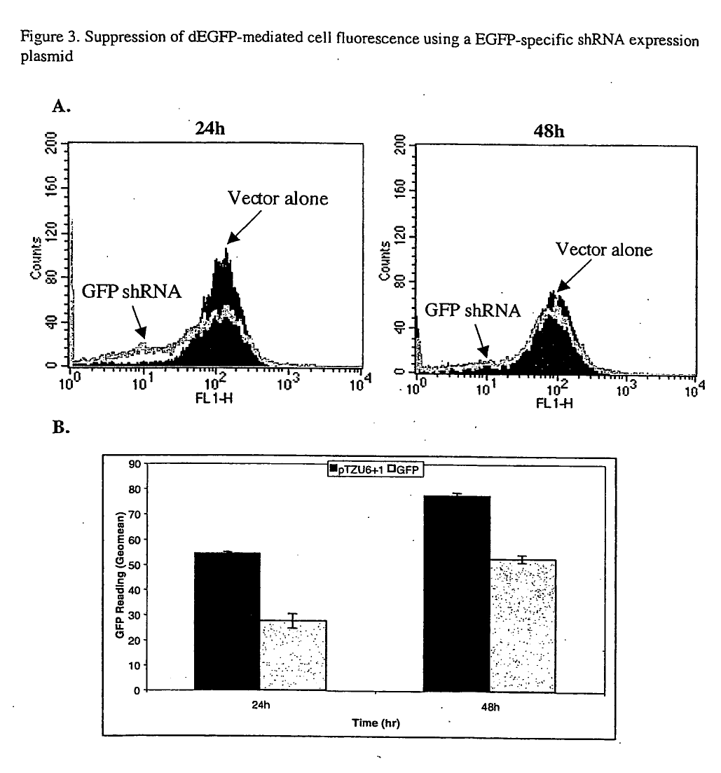

were annealed, and cloned into the SalI and XbaI sites present in pTZ(U6+1). DNA sequence analysis confirmed the presence of the EGFP shRNA insert within the SalI and XbaI sites. The pTZ(U6+1)GFP shRNA plasmid was tested by transient transfection in HEK 293 cells stably expressing the dEGFP gene. A total 2 μg of plasmid (either vector alone or pTZ(U6+1)GFP) were delivered in triplicate using Lipofectamine 2000. Cells were harvested at 24 h and 48 h post-transfection and assayed for dEGFP expression using FACS analysis (FIG. 3). This analysis indicated that the pTZ(U6+1)GFP plasmid, encoding the EGFP-specific shRNA, reduced dEGFP-mediated cell fluorescence by 40% at 24 h and 30% at 48 h. The observation of partial suppression was most likely due to transfection of only a subset of the target cells. This is exemplified by the presence of a second lower fluorescent peak in the histograms of cells receiving the pTZ(U6+1)GFP plasmid.

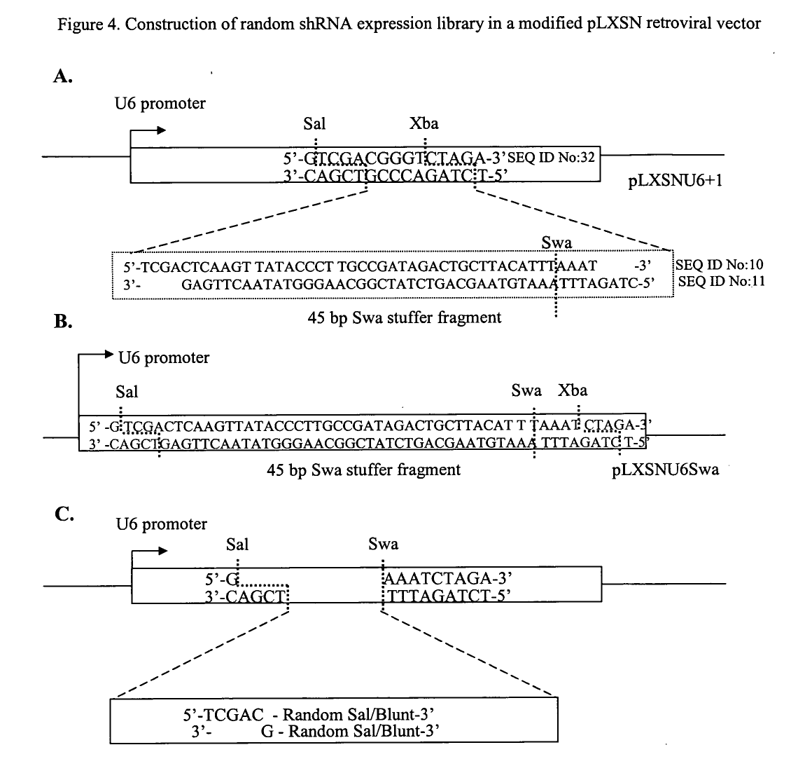

The U6+1 promoter contained in pTZ(U6+1) was PCR-amplified using the following forward and reverse primers: 5′-GCGCCTCGAGATAGGGAATTCGAGCTCGGTA-3′ (SEQ ID NO:8) and 5′-GCGCGGATCCTTGTAAACGACGGCCAGTGC-3′(SEQ ID NO:9). Following digestion with XhoI and BamHI, this DNA fragment was ligated into the multiple cloning site of the retroviral vector pLXSN to produce pLXSN(U6+1). To test this vector system for expression of effective shRNAs, the insert encoding the EGFP-specific shRNA was cloned into the SalI site located downstream of the U6+1 promoter. To further prepare this vector for use in construction of a random shRNA expression library, a stuffer fragment containing a SwaI site was inserted between the SalI and XbaI sites located 3′ to the U6+1 promoter to produce pLXSNU6Swa (FIG. 4). To accomplish this the following oligonucleotides were annealed and ligated into pLXSN(U6+1) previously digested with SalI and XbaI: 5′-TCGACTCAAGTTATACCCTTGCCGATAGACTGCTTACATTTAAAT-3′ (SEQ ID NO:10) and 5′-CTAGATTTAAATGTAAGCAGTCTATCGGCAAGGGTATAACTTGAG-3′(SEQ ID NO:11). DNA inserts encoding random shRNAs were digested with SalI and ligated into SalI-SwaI-digested pLXSNU6Swa.

### Example 2

### Random SiRNA Library

The following describes the methodology developed for generating random siRNA inserts and testing gene-expressed siRNAs for suppressing specific gene expression. In addition, the construction of random siRNA expression libraries using convergent promoters is outlined. To develop the method for generating inserts encoding short complementary sense and antisense RNAs, we used the p53 gene as a target. The following single-stranded oligonucleotide (63 bases) was synthesised containing a primer-binding site, SalI restriction site, five adenosines, 19 nucleotides specific to p53, five thymidines, a XbaI restriction site, and a second primer-binding site:

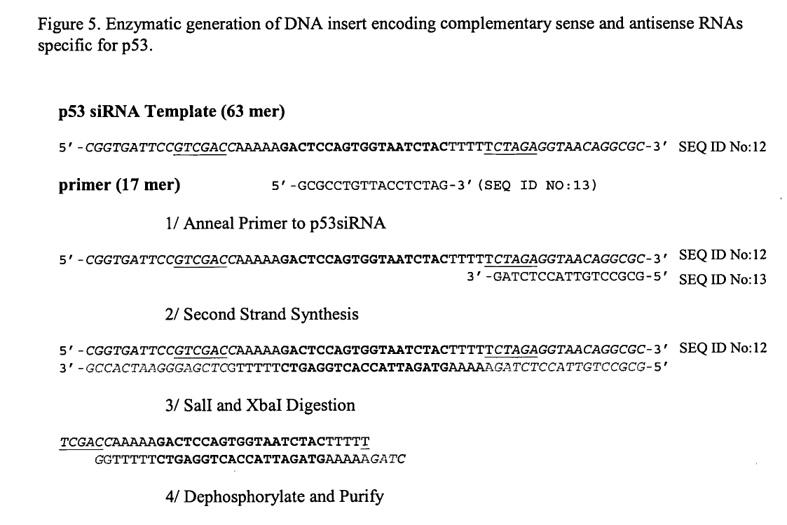

5′-CGGTGATTCCGTCGACCAAAAAGACTCCAGTGGTAATCTACTTTTTCTAGAGGTAACAGGCGC-3′ (SEQ ID NO:12)(FIG. 5).

A DNA primer (5′-GCGCCTGTTACCTCTAG-3′) (SEQ ID NO:13) was annealed to the above oligonucleotide and second-strand synthesis performed using Klenow DNA polymerase. Following generation of double-stranded DNA, this fragment was digested with SalI and XbaI and ligated into an appropriate vector containing convergent RNA polymerase III promoters.

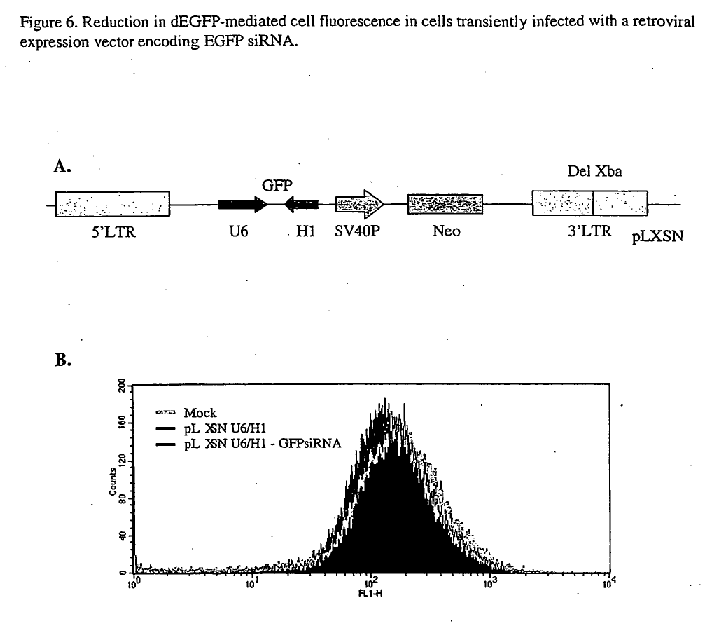

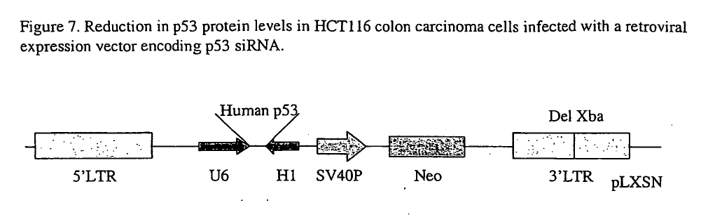

To establish a vector system in which convergent promoters drive the expression of short complementary RNAs, and there are no repeat sequences, we modified the pLXSN retroviral vector to include convergent U6 snRNA and H1 RNA polymerase III promoters (FIG. 6). The H1 promoter region was PCR-amplified from pSilencer using the primers 5′-GCCTGCAGGATATTTGCATGTCGCTATGTTCTGG-3′ (SEQ ID NO:14) and 5′-GCTCTAGAGAGTGGTCTCATACAGAACTTATAAG-3′ (SEQ ID NO:15), XbaI and Sbf1 digested, and inserted into the pLXSN(U6+1) vector. DNA sequence analysis confirmed that the U6 and H1 promoters were present and convergent in pLXSNU6/H1. To test this vector for its ability to induce RNAi in mammalian cells, we constructed siRNA expression vectors specific for EGFP (FIG. 6A) and human p53 genes (FIG. 7A). To construct the pLXSNU6/H1GFP vector, the oligonucleotides 5′-TCGACAAAAACGGCAAGCTGACCCTGAAGTTTTT-3′ (SEQ ID NO:16) and 5′-CTCAGAAAAACTTCAGGGTGAGCTTGCCGTTTTTG-3′ (SEQ ID NO:17) were annealed and cloned into the SalI and XbaI sites of pLXSNU6/H1 vector. The retroviral plasmid encoding GFPsiRNA (designated pLXSNU6/H1GFP) was transfected into Amphopack 293 packaging cells co-seeded with PG13 cells at a ratio of 10:1, respectively. Transfection efficiency approximated 40%. The virus-containing medium (VCM) was collected from these cells and used to infect HEK 293 or HCT116 target cells stably expressing EGFP. At 72 h post-infection, cells were harvested and examined for EGFP-mediated cell fluorescence using flow cytometry. This analysis indicated a minor reduction in cell fluorescence using this transient assay (FIG. 6B).

To test the effectiveness of the convergent retroviral expression system for regulating the expression of an endogenous gene, we constructed a derivative of pLXSNU6/H1 encoding complementary p53-specific sense and antisense RNAs. To this end, the following oligonucleotide was synthesised:

5′-CGGTGATTCCGTCGACCAAAAAGACTCCAGTGGTAATCTACTTTTTCTAGAGGTAACAGGCGC-3′ (SEQ ID NO:12)

The method described earlier for enzymatic generation of the second strand was performed and the DNA insert digested with SalI and XbaI and cloned between the U6 and H1 convergent promoters in pLXSNU6/H1 (FIG. 7A). The resulting plasmid, designated pLXSNU6/H1p53, was transfected into a 10:1 mixture of Amphopack 293 and PG13 packaging cells. The VCM was collected from these cells and used to infect HCT116 target cells. The infection efficiency approximated 63%, and infected cells were subjected to G418 (500 ug/ml) selection. The pooled population was harvested 8 days after selection and serially diluted to isolate single clones. Both the pooled population and single clones were monitored for p53 protein levels using Western analysis. This experiment demonstrated that p53 protein levels were reduced by at least 50% in the pooled cells. The gel illustrating this result shows three different concentrations of total protein lysates from HCT116 cells containing either the vector control (U6/H1) or the test vector (p53siRNA) probed for expression levels of p53 and β-actin. Analysis of selected clones indicated that the retroviral expression vector pLXSNU6/H1p53 reduced p53 protein levels. The gel illustrating this result shows total protein lysates from HCT116 clones containing either the control vector (U6/H1) or the test vector (p53siRNA) probed for levels of p53 and β-actin proteins.

To examine whether the gene-specific silencing mediated by pLXSNU6/H1p53 was occurring through RNAi, we examined the effect of treating selected HCT116 clones with Dicer-specific siRNA (described below). To this end, HCT116 clones containing either pLXSNU6/H1 (vector alone) or pLXSNU6/H1p53 were seeded at 5×10⁵ cells in a single well of a 6-well plate. The cells were allowed to recover for 24 h and then transfected with varying concentrations of Dicer siRNA (6 nM, 12 nM and 60 nM) or 60 nM of a nonsense siRNA (Dharmacon) using Lipofectamine 2000. After 3 h, the media was replaced with complete McCoys5A media. Cell pellets were harvested 24 h and 48 h post transfection and protein lysates were prepared for Western analyses of p53 and □β-actin protein levels. The steady-state level of p53 returned to wild type levels with increasing concentrations of Dicer siRNAs. The gel illustrating this result shows total protein lysates from HCT116 cells, containing either the vector control (U6/H1) or test vector (p53siRNA clone 8) and transfected with differing concentrations of Dicer siRNA, probed for levels of p53 and β-actin proteins. This reversal in reduction of p53 protein levels was not observed in HCT116 cells containing pLXSNU6/H1p53 and treated with the higher concentration of the nonsense siRNA. These results suggest that the observed suppression of p53 protein level by pLXSNU6/H1p53 is specific and dependent on Dicer, a key component of the RNAi mechanism in mammalian cells.

Given the above observations that the convergent U6-H1 promoter system, based in the retroviral expression vector pLXSN, was effective for inducing RNAi-mediated gene suppression in mammalian cells we proceeded to construct genome-wide siRNA expression libraries. Using the methodology established for the EGFP and p53-specific inserts, we synthesised the following oligonucleotide pool:

5′-CGGTGATTCCCTCGAGCAAAAANNNNNNNNNNNNNNNNNNNTTTTTCTAGAGGTAACAGGCGC-3′ (SEQ ID NO:18)

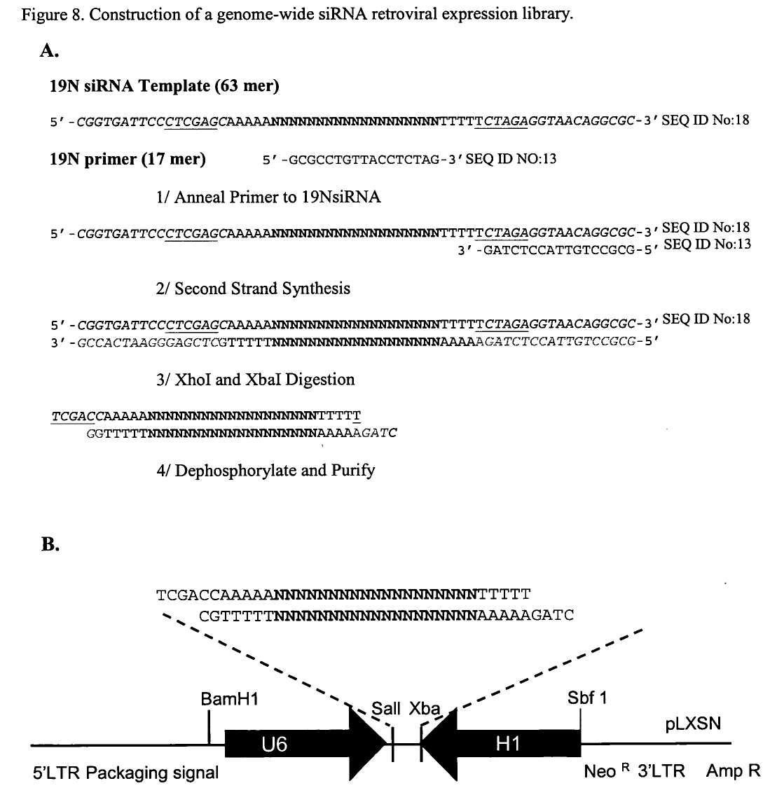

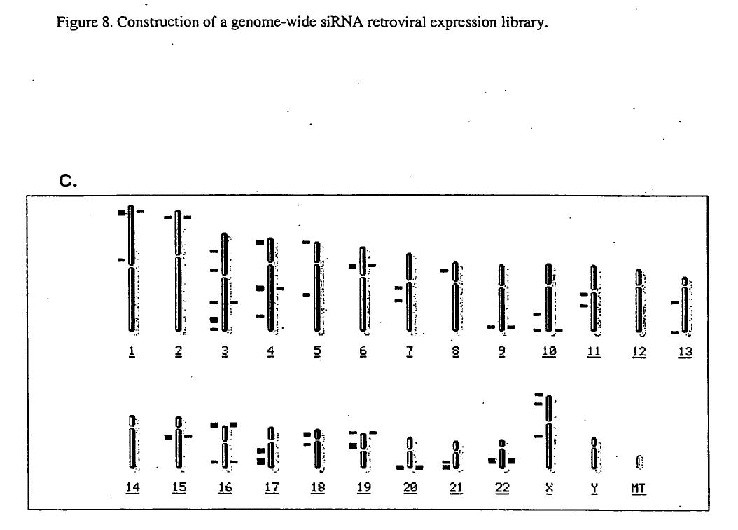

A total of 1 μmole of the above sequence (with 19 random nucleotides (N)) was synthesised using a special hand mix to ensure equimolar ratios of A, T, C and G (Integrated DNA Technologies, USA)534. A DNA primer (5′-GCGCCTGTTACCTCTAG-3′) (SEQ ID NO:13) was annealed to this pool of oligonucleotides and second-strand extension performed using Klenow DNA polymerase. Following this extension step, the DNA was digested with XhoI and XbaI and then dephosphorylated using calf intestinal alkaline phosphatase to prevent the generation of concatemeric inserts in the final expression library (FIG. 8A). The DNA inserts were gel-purified following electrophoresis on a non-denaturing 15% PAGE gel, excision of the 35 base pair fragments and extraction using the crush and soak method. The purified inserts were ligated in different insert to vector molar ratios (10:1 and 100:1) to 250 ng of the pLXSNU6/H1 vector pre-digested with SalI and XbaI. The vector was not dephosphorylated. Following overnight ligation at 16° C., the ligation was treated with SalI and the ligated products transformed into highly competent DH5□ bacterial cells. The transformed cells were either expanded as single clones or as liquid grown cells (FIG. 8B). In a 100 μl ligation volume, a total of 7.5×10⁵ clones were obtained with 70-90% of the plasmids containing inserts. DNA sequence analysis of inserts indicated random distribution of sequences when aligned to the human genome sequence (FIG. 8C).

### Example 3

### Constructs and SiRNAs

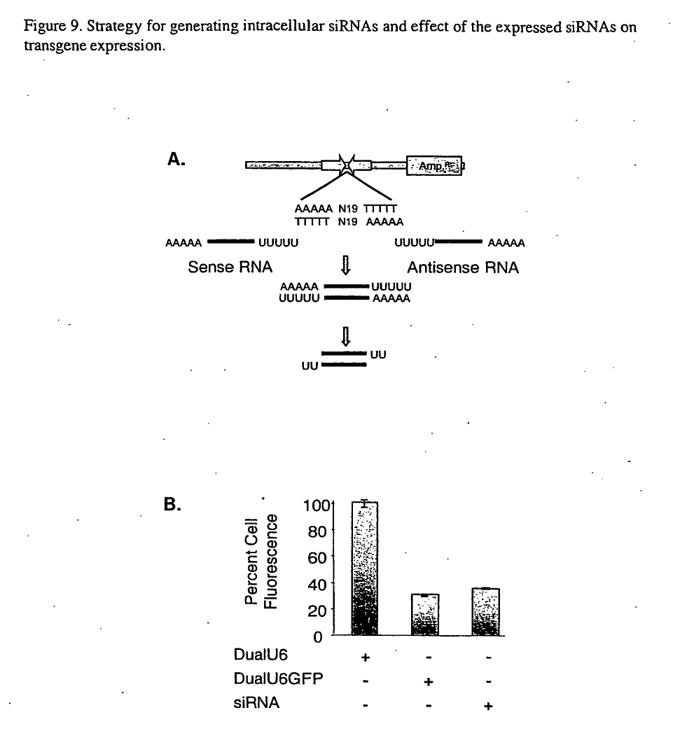

To develop a vector system for expressing siRNAs in mammalian cells compatible with generating RNAi for forward genetic selection, the convergent U6 promoter cassette indicated in FIG. 9A was designed. To determine the intracellular efficacy of this expression cassette for mediating specific gene silencing, the EGFP gene was used as a target.

To construct DualU6 containing convergent U6 promoters, the primers 5′-GCG CAA GCT TAT AGG GAA TTC GAG CTC GGT A-3′(SEQ ID NO:19), and 5′-GCG CTC TAG AGG TGT TTC GTC TCC ACA A 3′ (SEQ ID NO:20) were used to PCR amplify the U6+1 promoter region from pTZ(U6+1) (Paul, C. P., Good, P. D., Winer, I, and Engelke, D. R. (2002) Nature Biotech 20, 505-508) and the resulting amplicon cloned as a XbaI-HindIII fragment into pTZ(U6+1). The inserts encoding the sense and antisense RNAs were designed to include a 19 bp target-specific sequence (in bold below) flanked by two directional transcription terminators composed of five thymidines. The oligonucleotides used to construct DualU6GFP were 5′-TCGACAAAAACGGCAAGCTGACCCTGAAGTTTTT-3′ (SEQ ID NO:16) and 5′-CTAGAAAAACTTCAGGGTCAGCTTGCCGTTTTTG-3′ (SEQ ID NO:21), while the following were used to construct DualU6p53: 5′-TCGACAAAAAGACTCCAGTGGTAATCTACTTTTT-3′ (SEQ ID NO:22) and 5′-CTAGAAAAAGTAGATTACCACTGGAGTCTTTTTG-3′(SEQ ID NO:23). These oligonucleotides were synthesised (Sigma Genosys, Sydney, Australia), annealed and cloned into the SalI and XbaI sites of DualU6.

The RNA oligonucleotides used to form the siRNAs were synthesised by Dharmacon Research Inc (CO, USA) and the sequences were: GFP, 5′-CGGCAAGCUGACCCUGAAGdTdT (sense) (SEQ ID NO:24); p53(siRNA1), 5′-GACUCCAGUGGUAAUCUACdTdT (sense) (SEQ ID NO:25); and p53(siRNA2), 5′-GCAUGAACCGGAGGCCCAUdTdT (sense) (SEQ ID NO:26). These RNA oligonucleotides were annealed with corresponding antisense strands as described (Elbashir, S. M., Harborth, J., Lendeckel, W., Yalcin, A., Weber, K., and Tuschl, T. (2001) Nature 411(6836), 494-8).

### Example 4

### Effect of Expressed SiRNAs on Transgene Expression

Mammalian cells used in this study included the human embryonic kidney cell line EcR293 (Invitrogen, CA, USA) and the human breast cancer cell line MDA MB 231. The construction of the EcR293 cell line expressing the dEGFP gene has been described (Raponi, M., Dawes, I. W., and Arndt, G. M. (2000) Biotechniques 28, 840-844). EcR293 cells and their derivatives were maintained in DMEM containing 10% fetal calf serum supplemented with glutamine, streptomycin and penicillin. MDA MB 231 cells were grown in RPMI containing 10% fetal calf serum supplemented with glutamine.

Cells were seeded into 6 well plates 24 h prior to transfection. For all transfections, a total of 4 μg of plasmid DNA or 20 μM of siRNA was delivered using Lipofectamine 2000 (Invitrogen, CA, USA)) according to the manufacturer's instructions. Cells were harvested at 24 h and 48 h for flow cytometry analysis of EGFP expression (Becton Dickinson, USA). Fluorescent microscopy was performed using a fluorescence microscope (Nikon, Japan) with a B-2H filter cube.

A U6 convergent expression vector containing a EGFP-specific insert (DualU6GFP) was constructed and co-transfected with the pEGFP-N1 plasmid and the lacZ expression vector pSVβ□ into 293 embryonic kidney cells. Cells receiving DualU6GFP displayed a 40% reduction in cell fluorescence compared with cells transfected with the DualU6 control vector.

To further examine the utility of the dual U6 promoter, and the mechanism by which this vector regulated gene expression, the DualU6GFP plasmid was delivered to 293 cells containing a stably integrated destabilised EGFP (dEGFP) transgene. As shown in FIG. 9B, cells transfected with DualU6GFP displayed a reduction in dEGFP-mediated cell fluorescence with the level of reduction in fluorescence equal to that of the synthetic EGFP siRNA at 48 h post-transfection. Consistent with the requirement for expression of the sense and antisense RNAs from DualU6GFP, gene silencing via this vector displayed a 24 h delay compared with a synthetic siRNA targeted to the same region of the dEGFP mRNA. The reduction in cell fluorescence exhibited by cells containing the DualU6GFP plasmid was confirmed using fluorescence microscopy. This illustration shows the cell fluorescence in cells transfected with DualU6, DualU6GFP or GFP-specific siRNA. As with the synthetic siRNAs, the residual population displaying cell fluorescence most likely represents cells that have not been transfected with the expression plasmid.

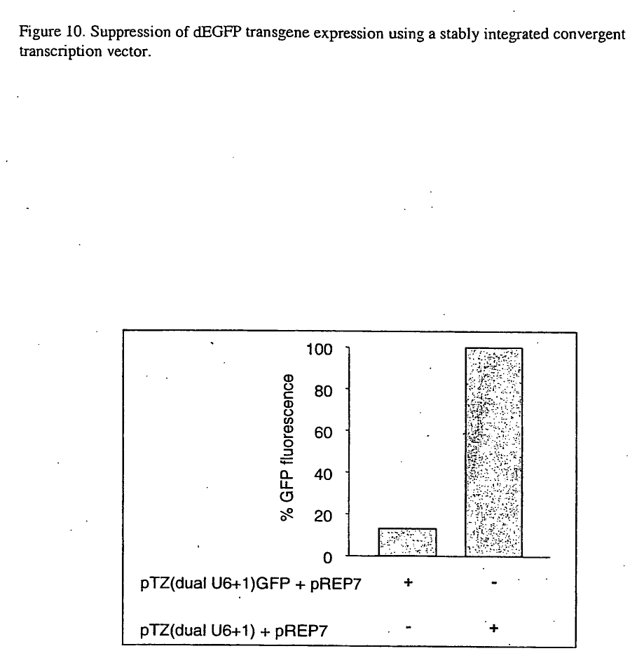

To examine the utility of the DualU6GFP expression system in long term regulation of gene expression in mammalian cells, either the pDualU6GFP plasmid, or the pDualU6 vector, was co-delivered with pREP7 (containing the marker conferring resistance to hygromycin) to HEK 293 cells expressing the dEGFP transgene. Following selection for cells stably maintaining the DualU6GFP plasmid, cells were examined for dEGFP-mediated cell fluorescence. As shown in FIG. 10, cells containing the DualU6GFP plasmid displayed a significant reduction in cell fluorescence compared with cells receiving the DualU6 control vector. This result indicates that the convergent expression cassette described can be used to mediate long term regulation of gene expression in mammalian cells.

It has been reported that shRNAs, or co-expression of small antisense and sense RNAs, produce specific gene silencing by processing to siRNAs. To determine the mechanism of action of the DualU6GFP expression system, transfected cells were examined for dEGFP protein levels, dEGFP mRNA levels and the presence or absence of small RNAs encoded by the U6 convergent expression vector containing an EGFP-specific insert.

Western analysis was performed as follows: cell lysates were prepared using RIPA buffer supplemented with protease inhibitors aprotonin (1 μg/ml), leupeptin (10 μg/ml) and DMSF (100 μg/ml). Total protein was loaded onto 4-12% Bis-Tris agarose gels (Invitrogen, CA, USA), separated by electrophoresis and transferred to polyvinylidene fluoride (PVDF) membrane. The antibodies used for detection of specific proteins in the Western analysis included: GFP, mouse polyclonal (Clontech), PKR monoclonal (Cell Signaling), PKR phospho rabbit polyclonal (Cell Signaling), p53 mouse monoclonal (Oncogene Research Products) or β-actin mouse monoclonal (Sigma) antibodies. Secondary antibody detection was performed using either the goat anti-mouse horseradish peroxidase (HRP)-linked or the goat anti-rabbit HRP (SantaCruz), followed by visualisation using the luminol/enhancer chemiluminescent substrate (Amersham Pharmacia Biotech, Piscataway, N.J.).

Western analysis showed that the dEGFP protein levels were reduced in cells expressing the siRNA from the U6 convergent expression vector and that this effect was specific. The level of suppression of the dEGFP protein was equivalent to that mediated by delivery of synthetic siRNAs. The gel illustrating this result shows the protein level of dEGFP and β-actin in HEK 293 cells (containing an integrated dEGFP gene) transfected with DualU6, DualU6GFP or the GFP-specific siRNA. An examination of dEGFP target mRNA levels indicated that both the synthetic siRNAs and those expressed from the U6 convergent plasmid reduced target mRNA. The gel illustrating this result shows the level of dEGFP mRNA and 18S rRNA in HEK 293 cells (containing an integrated dEGFP gene) transfected with DualU6, DualU6GFP or the GFP-specific siRNA. This latter result suggests that DualU6GFP produces siRNAs capable of mediating turnover of the target mRNA, an observation consistent with the mechanism of RNAi.

### Example 5

### Gene Suppression by Complementary RNAs Expressed from a U6 Convergent Cassette is Dicer-Dependent

To further confirm that the DualU6GFP plasmid maintains the potential to produce siRNAs, the transcripts expressed from this plasmid were identified using northern blot analysis. RNA for RNA analysis was isolated using Trizol (Invitrogen, CA, USA) and immobilised onto nylon membrane (Invitrogen, CA, US), for detection using standard probe hybridisation. For the detection of small antisense and sense RNAs encoded by DualU6GFP, the following oligonucleotides were end-labelled and hybridised to these membranes at 37° C. for 1 h: 5′-TCGACAAAAACGGCAAGCTGACCCTGAAGTTTTT-3′ (SEQ ID NO:16) or 5′-CTAGAAAAACTTCAGGGTCAGCTTGCCGTTTTTG-3′(SEQ ID NO:21). Membranes were analysed using a phosphorimager (Molecular Dynamics, USA) and an ImageQuant software package (Molecular Dynamics, USA).

Bands of the expected length were observed only in cells containing the DualU6GFP plasmid and not in vector controls. In addition, using strand-specific probes, it was possible to show that within the cells containing the U6 convergent EGFP vector both the antisense and sense RNAs were present. The sizes of the transcripts confirmed that the directional terminators were operative and that U6-directed transcriptional machinery efficiently truncated the antisense and sense transcripts within the convergent transcription unit. The gel illustrating this result shows the level of sense and antisense small RNAs encoded by the DualU6GFP plasmid. It also shows the absence of these small RNAs in mock-transfected cells and cells transfected with the DualU6 control vector. The above results indicate that the use of U6 convergent promoters in a single expression cassette can produce sense and antisense RNAs that mediate specific gene suppression in a manner consistent with RNAi.

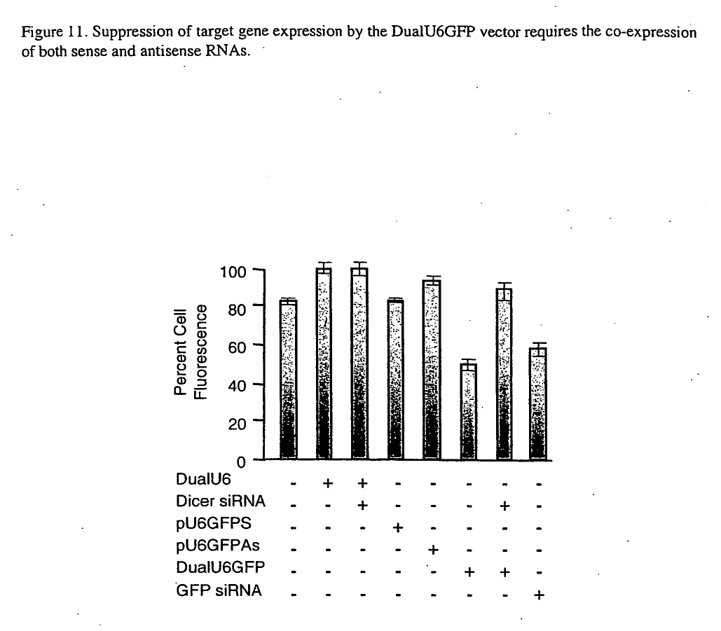

To demonstrate the necessity for convergent U6 promoters in the DualU6GFP vector, and therefore the expression of both sense and antisense RNAs, to mediate suppression of the dEGFP target gene, derivatives of this plasmid containing only a single U6 promoter were constructed. These vectors were designated pU6GFPS and pU6GFPAs and were expected to encode small sense and antisense EGFP RNAs under control of the U6 promoter, respectively. Each of these plasmids was used to transiently transfect 293 cells expressing the dEGFP transgene. Cell populations were then analysed for dEGFP-mediated cell fluorescence. This analysis indicated that the expression of either sense or antisense EGFP strands alone was insufficient to suppress the dEGFP gene, and that full inhibition of this target gene required the co-expression of both strands within the same cell (FIG. 11).

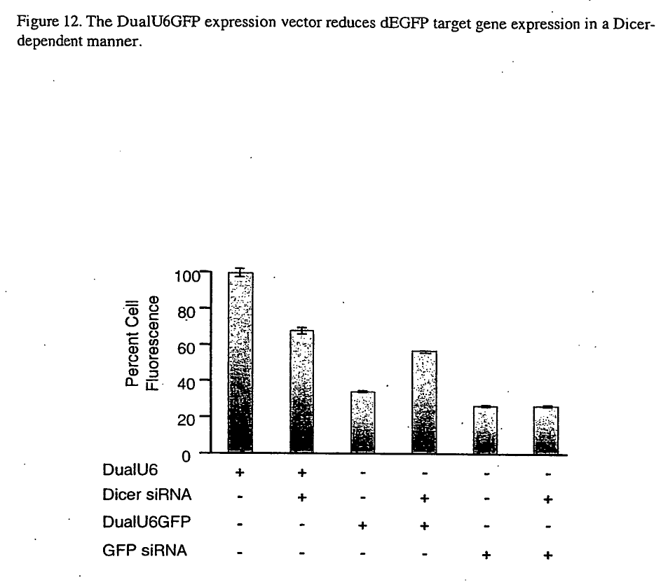

Given that the cells co-expressing the sense and antisense EGFP RNAs displayed many of the hallmarks of RNAi, the issue of whether gene silencing occurred through formation of dsRNA was determined. Toward this end, the Dicer siRNA was utilised as a tool to determine if the observed suppression was Dicer-dependent (Hutvagner et al (2001) Science 293, 834-838). 293 cells expressing the dEGFP transgene were transfected with DualU6GFP in the presence and absence of the synthetic siRNA specific for Dicer. As shown in FIG. 12, the Dicer siRNA completely reversed the reduction in cell fluorescence mediated by the EGFP-specific U6 convergent plasmid. In contrast, cells transfected with both the synthetic EGFP- and Dicer-specific siRNAs still displayed a reduction of cell fluorescence, as the mechanism of synthetic siRNAs is Dicer-independent. These results suggest that the small sense and antisense RNAs encoded by DualU6GFP anneal to form dsRNA that is processed by Dicer into authentic siRNAs. It is most likely that gene silencing is then directed by these processed siRNAs.

It has been proposed that dsRNA greater than 30 base pairs in size induce a global response that results in activation of the double-stranded RNA-specific protein kinase PKR (Paddison, P., Caudy, A. A., and Hannon, G. J. (2002) Proc. Natl Acad. Sci. 99, 1443-1448). To eliminate PKR activation as being responsible for the gene silencing observed using this unique expression system, the levels of both total PKR and activated PKR were examined in 293 cells receiving the DualU6GFP plasmid. This analysis indicated that co-expression of the sense and antisense EGFP RNAs and formation of dsRNAs did not activate PKR, suggesting that the observed gene silencing effect was specific and not related to this global response mechanism. The gel illustrating this result shows the level of PKR, activated PKR and β-actin in cells transfected with the DualU6 control vector, DualU6GFP or the GFP-specific siRNA.

### Example 6

### Suppression of p53 Protein Levels Using a Convergent U6 Expression Vector

Whether the U6 convergent promoter system could be used to control the expression of endogenous genes in mammalian cells was determined. For this purpose, the TP53 gene that encodes the p53 tumor suppressor protein was chosen as a target. To this end, a U6 convergent expression vector was constructed containing an insert encoding a p53-specific siRNA. The target site selected was identical to that reported earlier for synthetic p53-specific siRNAs (Brummelkamp, T. R., Bernards, R., and Agami, R. (2002) Science 296, 550-553). The p53-specific U6 convergent expression plasmid, DualU6p53, was transfected into MDA MB 231 breast cancer and 293 cells and cells were harvested and analysed for p53 protein levels out to 120 h post-transfection. Delivery of the DualU6p53 plasmid resulted in a significant and specific reduction of p53 protein. The gel illustrating this result shows the level of p53 and β-actin proteins in cells transfected with DualU6, DualU6p53 or p53-specific siRNAs. This result indicates that the U6 convergent promoter system can be used to effectively suppress the expression of endogenous genes through RNAi in mammalian cells.

### Example 7

### Convergent Transcription Induces Stable Suppression of Endogenous Gene Expression

As described above, we have shown that the DualU6GFP expression vector can be used to regulate EGFP gene expression both in transient assays and stably selected pooled populations. In addition, we demonstrated that the endogenous p53 gene is suppressible by DualU6p53 in transiently transfected HEK 293 cells. In this example we co-delivered the DualU6p53 plasmid with pREP7 (containing the hygromycin resistance gene) to HEK 293 cells containing the dEGFP transgene. In addition, we also co-transfected these same cells with the DualU6GFP and pREP7. Each of these populations and the vector alone with pREP7 were exposed to 500 μg/ml hygromycin for two weeks. Stable cells were selected and examined for p53 protein levels by Western analysis. The analysis indicated that cells containing the DualU6p53 plasmid showed a significant reduction in p53 levels compared with cells receiving the control vector or cells containing DualU6GFP. The gel illustrating this result shows the level of p53 and β-actin proteins in cells stably transfected with DualU6, DualU6GFP or DualU6p53 constructs. This suggests that the observed suppression is sequence-specific and that long term regulation of endogenous gene expression can be achieved in mammalian cells using convergent transcription.

### Example 8

### p53SiRNA Spiked Library

### Chemotherapeutic Drug Resistance Screen

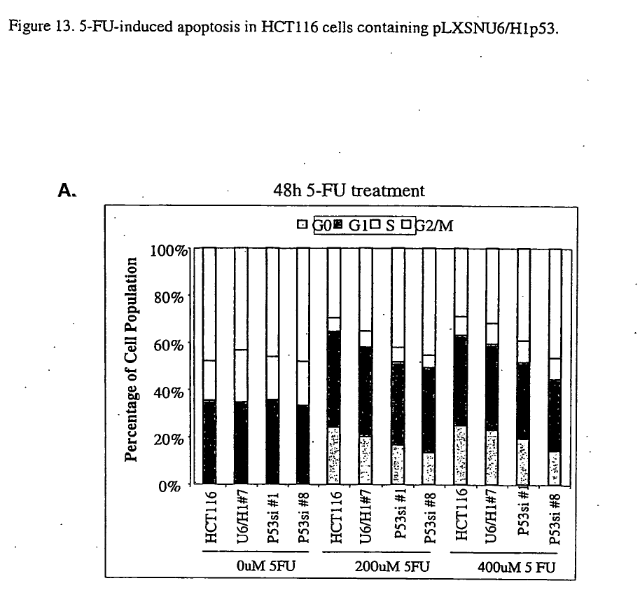

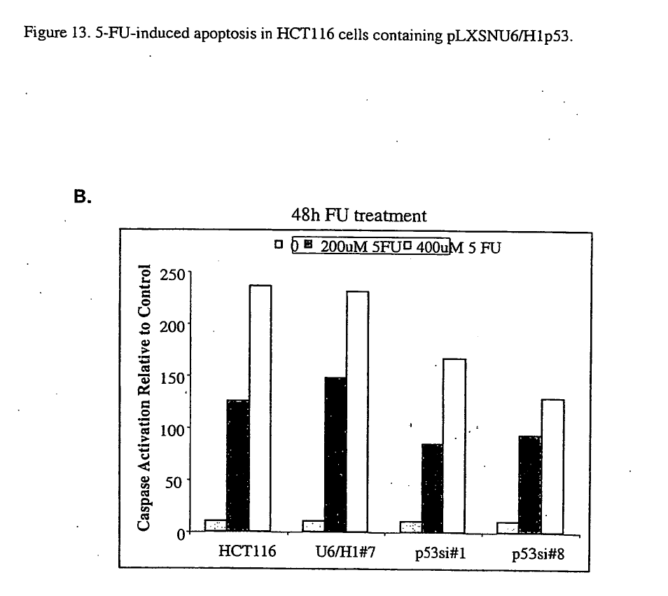

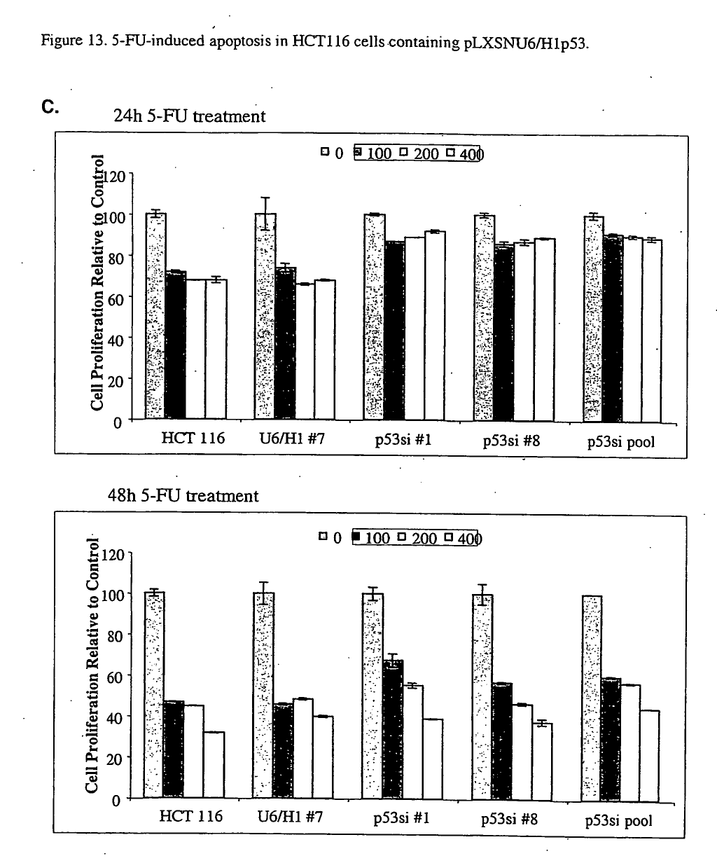

To examine the utility of genome-wide RNAi libraries for forward genetic selection in mammalian cells, we performed two experiments. In the first, we generated HCT116 clones containing pLXSNU6/H1 or pLXSNU6/H1p53 and showed that convergent transcription of the p53 sequence in the latter suppressed p53 protein levels. These clones were further characterised for their cellular responses to the chemotherapeutic agent 5-fluorouracil (5-FU). It has been shown that mutations in p53 result in cellular resistance to 5-FU-induced apoptosis (Bunz, F. et al (1999) J Clinical Investigation 104: 263-269). Clones containing pLXSNU6/H1 or pLXSNU6/H1p53 were seeded at either 2.5×10⁵ cells per well of a 6 well plate (to examine subG1 and caspase activation) or 1×10⁴ cells per well of a 96 well plate (for examining cell proliferation and viability). Cells were allowed to recover for 24 h and then treated with varying concentrations of 5-FU (100 uM, 200 uM and 400 uM) for 24 h. At this point, cells were examined for cell cycle distribution using propidium iodide (PI) staining, induction of apoptosis using caspase activation assay and cell viability using the MTT cell proliferation assay (FIG. 13). Cells expressing the p53-specific siRNA showed decreases in subG1 cells and caspase activity compared with control cells (FIGS. 13A and B). In addition, the cells suppressed for p53 protein levels using pLXSNU6/H1p53 displayed increased cell survival in an MTT assay (FIG. 13C). Upon demonstration of a differential response of the clones containing either pLXSNU6/H1 or pLXSNU6/H1p53 to 5-FU-induced apoptosis, cells from the latter are diluted in a larger background of cells containing pLXSNU6/H1. These mixed cell populations are seeded at 2×10⁶ cells per T150 flask. Following 24 h recovery, cells are exposed to 400 μM 5-FU for 18 h, re-seeded at a 4×10⁵ cells per 150 mm dish and allowed to form colonies for 10-14 days in the absence of 5-FU. Analysis of the 5-FU resistant clones indicates an enrichment of clones containing pLXSNU6/H1p53.

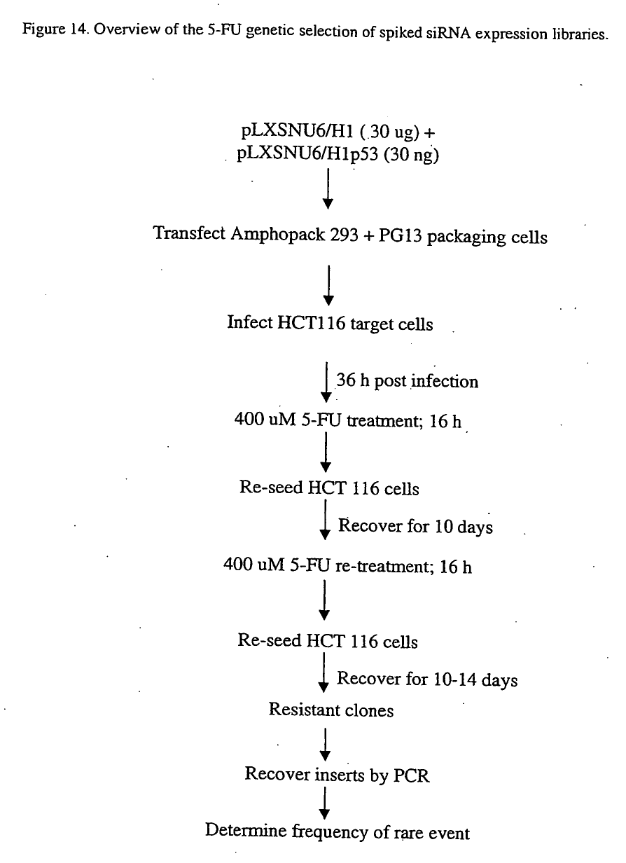

In the second experiment we constructed expression libraries in which the pLXSNU6p53 retroviral vector was spiked into a larger background of vector alone and then screened in HCT116 cells for the enrichment of pLXSNU6/H1p53 using genetic selection (FIG. 14). The vector pLXSNU6/H1p53 was diluted 1:10³ and 1:10⁴ in pLXSNU6/H1 and this DNA mix used to transfect a 7:1 mixture of Amphopack 293 and PG13 packaging cells. To this end, 2×10⁶ AmphoPack 293 cells and 3×10⁵ PG13 cells were seeded in 10 T75 culture flasks for both the 1:10³ and 1:10⁴ libraries. In addition, flasks were also established for pLXSNU6/H1 (vector control), pLXSNU6/H1p53 (positive control), and pLXSNGFP (as a indicator of transfection efficiency). At 48 h following seeding, the cells were treated with 180 μM calcium phosphate containing 5 mM butyrate and 50 μM chloroquine with or without DNA. In the case of the libraries, a total of 30 μg of reconstituted DNA (for example, 30 ng of pLXSNU6/H1p53 plus 30 ug of pLXSNU6/H1 for the 1:10³ library) was delivered. After 8 hr incubation the transfection solution was replaced with complete DMEM medium and cells allowed to recover for 24 h. After this period, the media on the packaging cells was again replaced with 15 ml complete DMEM medium supplemented with 1 mM sodium pyruvate, from which the VCM was collected after 16 h. The VCM from 10×T75 flasks were pooled, filtered through a 0.45 μM filter and combined with 5 μg/ml polybrene. This VCM was placed on 10×T150 flask of HCT116 cells for 24 h, after which the VCM was replaced with McCoys5A medium. The target HCT116 cells were initially seeded at 2.5×10⁶ cells per T150 flasks 36 h prior to infection and a total of 10 flasks were used. The infection efficiency obtained using these conditions was at least 40%. At 36 h post-transduction, the HCT116 cells reached 60% confluence. At this point, the media was changed to McCoys5A containing 400 μM 5-FU. The cells were exposed to 5-FU for 16 h, after which they were harvested, pooled and re-seeded at 3.5×10⁶ cells per T150 flask. Following 10 days of growth in the absence of 5-FU, cells were re-exposed to 400 uM 5-FU for 16 h, harvested and seeded at 4×10⁶ cells per 150 mm dish. These cells are allowed to form colonies over 10 to 14 days at which point independent colonies are characterised for the presence of pLXSNU6/H1 or pLXSNU6/H1p53 proviral DNA. This analysis indicates that selection in the presence of 5-FU results in a significant enrichment in resistant colonies harbouring the pLXSNU6/H1p53 vector. This result would suggest that random RNAi expression libraries, based around the convergent transcription expression cassettes described in this application, can be used in forward genetic selections in mammalian cells to identify relevant genetic inhibitors (and therefore target genes).

### Example 9

### Additional Retroviral Expression Vector Systems

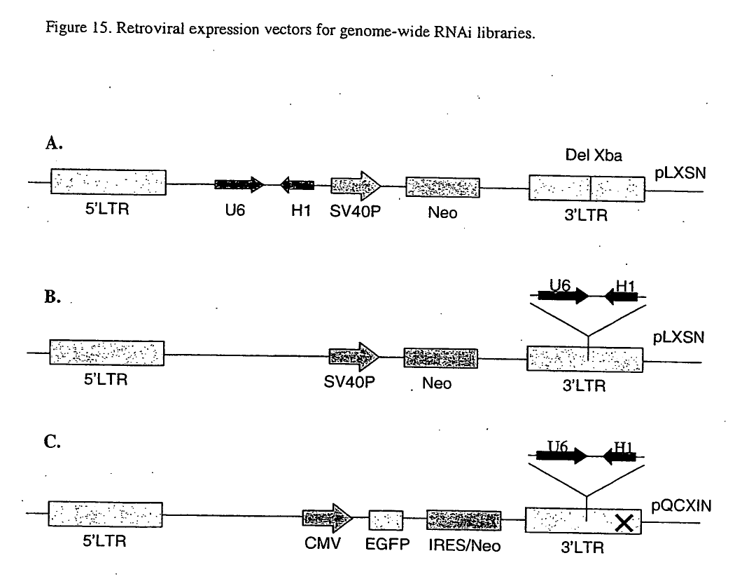

A variety of retroviral expression vectors can be used for the expression of genetic inhibitors, such as shRNAs, and the over-expression of specific genes. To extend the utility and applicability of the genome-wide RNAi expression libraries described in this invention, we have also constructed alternative retroviral expression vectors (FIG. 15). The vector pLXSNU6/H1 has been described earlier and contains the convergent U6-H1 promoter cassette in the multiple cloning site of pLXSN (FIG. 15A). In this vector system, the 5′LTR remains transcriptionally active upon proviral DNA integration and the U6-H1 cassette is located between the 5′ and 3′LTRs. This vector also contains a NeoR gene that permits selection of cells containing the integrated retroviral vector using the agent G418. One alternative vector system illustrated in FIG. 15B contains the U6-H1 expression cassette located in the 3′LTR. To construct this vector, the 3′LTR was first removed from pLXSN and subcloned into pSP72 as a AflIII-EcoRI fragment to produce pSP72LTR. The U6-H1 cassette was then PCR-amplified using the following PCR primers:

### 5′-GCGCTAGCCGTTAACTCGAGGATCCAAGGTCG-3′ (SEQ ID NO:27) and

### 5′-GCGCTAGCCACAGCCGGATCCTTGTAAACGAC-3′ (SEQ ID NO:28).

The PCR amplicon is digested with NheI and subcloned into the unique NheI site located within the 3′ LTR in pSP72LTR. Following insertion of these sequences, the 3′LTR containing the U6-H1 convergent promoters are subcloned as an AflIII-EcoRI fragment back into pLXSN to replace the wild type 3′LTR. The end result is the positioning of the U6-H1 convergent promoter cassette in the 3′LTR region. Upon infection and proviral integration this cassette will be copied as part of the 5′LTR resulting in two copies of the cassette, one of which will be located upstream of the transcription start site in the 5′LTR. The other form of the retroviral expression vector is shown in FIG. 15C. In this scenario, a self-inactivating retroviral construct, designated pQCXIN, is used as the starting material. The XbaI site located in the 3′LTR is removed by XbaI digestion, end-filling and re-ligation. The U6-H1 fragment is PCR-amplified using the following PCR primers: 5′-GCGCTAGCCGTTAACTCGAGGATCCAAGGTCG-3′ (SEQ ID NO:27) and 5′-GCGCTCGAGCACAGCCGGATCCTTGTAAACGAC-3′(SEQ ID NO:29). The DNA fragment is then digested with XhoI and subcloned into the unique SalI site located in the 3′LTR. In addition, the EGFP open reading frame (including a Kozak consensus sequence) is PCR-amplified from pEGFP-N1 using the following PCR primers: 5′-GCAGTCGACGGTACCGCGGGCCCGGTCGC-3′ (SEQ ID NO:30) and 5′-GGAATTCGCGGCCGCTTTACTTGTACAGC-3′ (SEQ ID NO:31). Following digestion with BamH1 and EcoR1, this fragment is subcloned into the multiple cloning site in the modified pQCXIN vector. The end vector will contain the EGFP and NeoR markers and the U6-H1 expression cassette. Furthermore, this vector system will produce two copies of the U6-H1 cassette upon proviral DNA integration and with no transcription directed from the 5′LTR.

### Example 10

### Construction of Target Gene and Genome (Viral, Pathogen)-Specific ShRNA and SiRNA Libraries

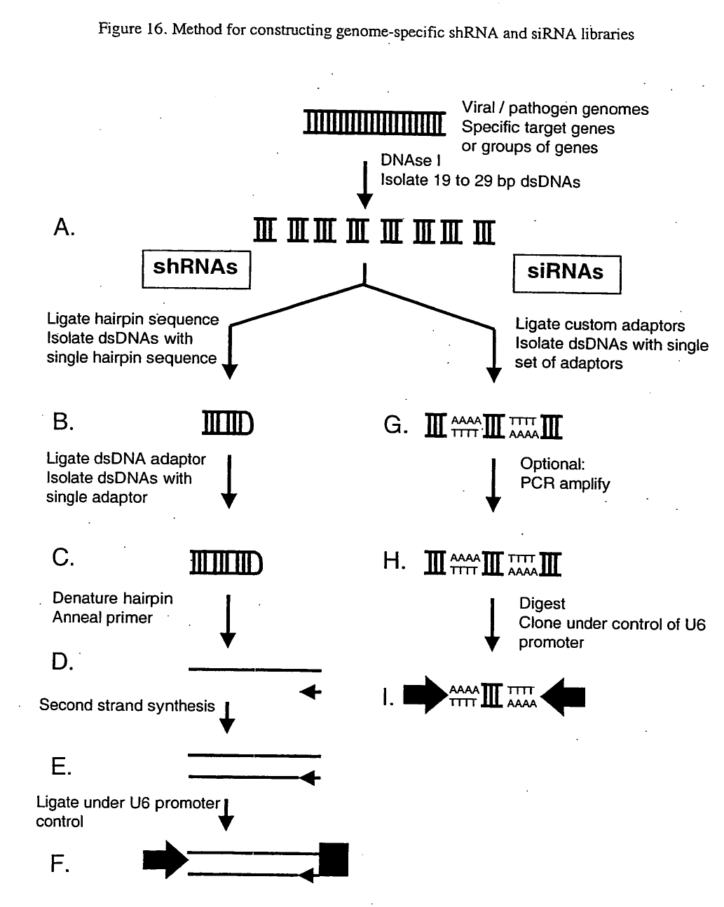

The strategies described above allow the production of RNAi expression libraries that contain dsRNA genetic inhibitors for each of the expressed genes of any genome including mammalian cells. These same libraries also have utility for identifying both host genes and viral or pathogen-derived genes that play a major role in the susceptibility of cells to infection by viruses and pathogens. The described methods can be modified to construct RNAi expression libraries restricted to a specific viral or pathogen genome or to a limited number of targets genes. The latter application is particularly relevant for probing gene function of up- or down-regulated genes identified in large-scale microarray or subtractive hybridisation experiments where only a subset of genes is implicated in the phenotype under investigation. FIG. 16 summarises the strategy for constructing target gene(s) and genome-specific shRNA and siRNA expression libraries. In the initial step, the target gene(s) or viral or pathogenic genome is treated with DNAseI to fragment the starting DNA into 19-29 bp fragments (FIG. 16A). To construct a shRNA expression library, the pool of DNA fragments is ligated to a universal hairpin sequence and all DNA fragments containing a single hairpin linker are isolated (FIG. 16B). A dsDNA adaptor (containing a primer-binding site) is then ligated to the end of these DNAs (that does not contain the hairpin linker) and all fragments having a single hairpin linker and dsDNA adaptor are isolated (FIG. 16C). This pool of DNA is then denatured, annealed to the universal primer, subjected to second-strand synthesis and then digested and ligated under control of the U6 promoter in a mammalian expression plasmid (FIG. 16D-F). To construct a siRNA expression library, the randomly fragmented 19-29 bp DNAs are ligated to a dsDNA adaptor which includes a 3′ sequence of at least four adenosine residues and all DNAs containing a single set of adaptors are isolated (FIG. 16G). These DNAs can either be PCR-amplified using a primer specific for the ligated adaptors (FIG. 16H) or digested directly and ligated between convergent U6 promoters (FIG. 16I).

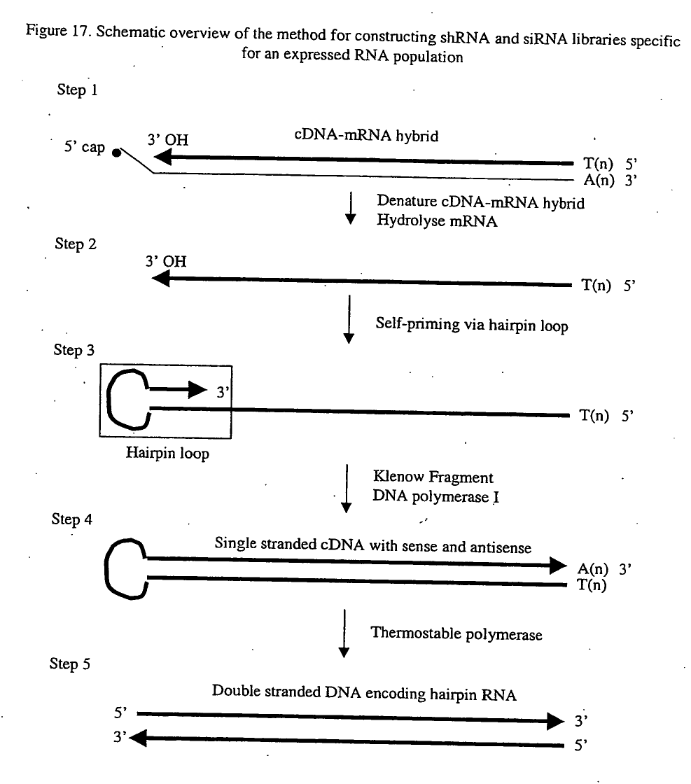

The described methods can also be modified to construct RNAi libraries specific for the expressed RNA population in specific cell types or tissues. An outline of this approach is shown in FIG. 17. To construct this library, the phenomenon of self priming during cDNA synthesis is used. During the synthesis of the first strand of cDNA using AMV reverse transcriptase, the 3′ termini of single-stranded cDNA can form hairpin structures due to concomitant degradation of the template RNA (Steps 1 and 2). Transient formation of these hairpin structures provides a priming point for reverse transcriptase to initiate second strand synthesis (Step 3). This intramolecular dsDNA molecule (Step 4) is converted into an intermolecular dsDNA fragment by second strand synthesis using high temperature (to denature the template) and thermostable DNA polymerase (Step 5). The end result is the production of DNA inserts encoding long inverted repeat RNA sequences capable of forming dsRNA. In the case of long dsRNAs, these could be targeted for maintenance within the nucleus using 5′ decapping recognition sequences and a cis-acting hammerhead ribozyme. Alternatively, the resulting DNA fragments could be subjected to the method described in FIG. 16 to generate siRNA or shRNA expression libraries. All of these libraries would be specific for the expressed gene set contained within a certain cell type or tissues.

### Example 11

### Identification of HIV Therapeutics Using HIV-Derived ShRNA Libraries

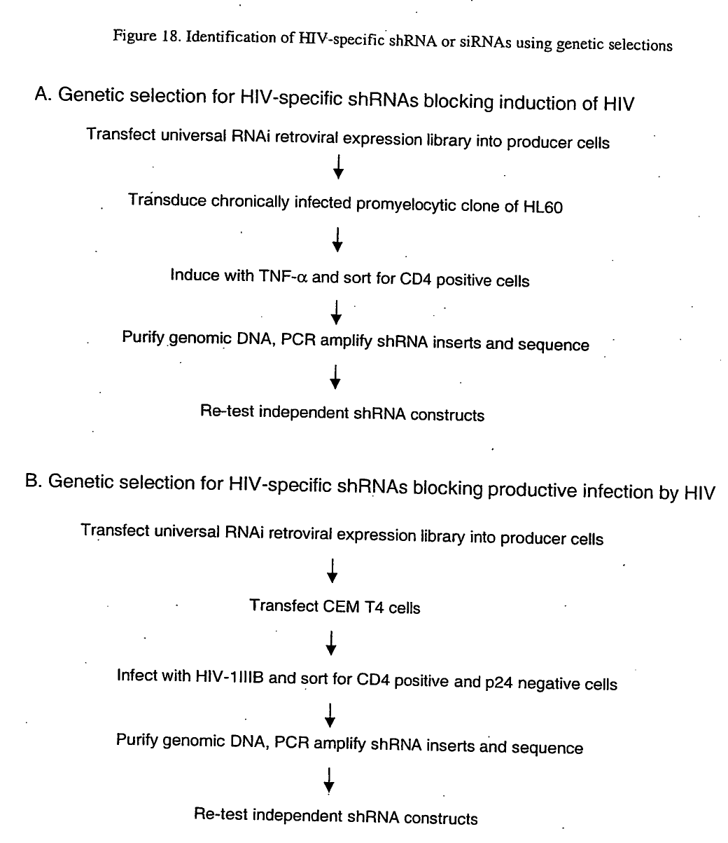

Genetic selection assays can be used to screen a HIV-specific RNAi expression library for effective RNAi construct that confer resistance to HIV infection or that interfere with the productive or latent phases of the viral life cycle. Such genetic selection assays using genetic suppressor element libraries have been described (Dunn, S. J., Park, S. W., Sharma, V., Raghu, G., Simone, J. M., Tavassoli, R., Young, L. M., Ortega, M. A., Pan, C-H., Alegre, G. J., Roninson, I. B., Lipkina, G., Dayn, A., and Holzmayer, T. A. (1999) Gene Therapy 6, 130-137) and are outlined in FIG. 18. In one assay, chronically infected promyelocyctic HL60 cells, which are 99% CD4 positive until induction of latent HIV, can be induced to lose CD4 upon the addition of TNFα (type 4) (FIG. 18A). Expression of an effective HIV-specific shRNA will be expected to interfere with this induction and result in the retention of CD4 on the cell surface. Cells containing effective shRNA constructs can then be separated from the CD4-negative population using FACs sorting. These constructs should be effective at inhibiting HIV induction in latently infected cells. In a second assay, CEM T4 cells infected with replicating HIV display an accumulation of p24 and a reduction of CD4 (FIG. 17B). Thus, expression of an effective shRNA construct that interferes with productive infection can be identified by enriching for cells exhibiting the CD4-positive and p24-negative phenotype using FACs. Both of these genetic selection systems can identify novel HIV-specific shRNA expressing vectors that could be used as gene therapy against multiple stages of the HIV life cycle.

The system described provides a novel alternative expression modality to shRNA-expressing plasmids for gene silencing in mammalian cells. The convergent promoter system also provides a basis for generating randomised RNAi libraries in which random double-stranded DNA oligonucleotides can be introduced between the convergent U6 promoters. The expansion of this design to include two different RNA polymerase III promoters in opposing orientations, or combinations of RNA polymerase II and/or III promoters, with random oligonucleotide sequences between the convergent promoters, would produce a randomised RNAi library expressing functional siRNAs in mammalian cells and containing no inverted repeat sequences. Such genome-wide RNAi libraries would be useful for performing forward genetic screens similar to those reported using randomised ribozyme libraries (Kawasaki, H., Onuki, R., Suyama, E. and Taira, K. (2002) Nature Biotech 20:376-380) and universal peptide libraries (Xu, X., Leo, C., Jang, Y., Chan, E., Padilla, D., Huang, B. C. B., Lin, T., Gururaja, T., Hitoshi, Y., Lorens, J. B., Anderson, D. C., Sikic, B., Luo, Y., Payan, D. G. and Nolan, G. P. (2001) Nature Genetics 21:23-29). A significant advantage in using randomised RNAi libraries, over other nucleic acid-based libraries, in forward genetic approaches in mammalian cells would be the identification of 21 bases of complete sequence complementarity to the intracellular target RNA that is linked to the modified cellular phenotype. This length of sequence conservation could be used to more effectively identify candidate genes using homology-based search tools. In addition, these sequences could be chemically synthesised and used as tools for further validation of the identified targets or as potential therapeutics.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Ausbel F M et al (1989) Current Protocols in Molecular Biology, John Wiley & Sons, New York N.Y.
Baker, M. W., and Macagno, E. R. (2000) Curr Biol 10(17), 1071-4.
Baulcombe, D. C. (1996) Plant Mol Biol 32(1-2), 79-88.
Beaucage and Caruthers (1981) Tetrahedron Letts. 22(20):1859-1862
Berkow et al eds., (1987) The Merck Manual of Diagnosis and Therapy, 15th Ed., Rahway, N.J., pp 1206-1228.
Billy, E., Brondani, V., Zhang, H., Muller, U. and Filipowicz, W. (2001) Proc. Natl Acad Sci 98, 14428-14483.
Brummelkamp, T. R., Bernards, R., and Agami, R. (2002) Science 296, 550-553.
Caplen, N. J., Parrish, S., Imani, F., Fire, A., and Morgan, R. A. (2001) Proc. Natl. Acad. Sci. 98, 9742-9747.
Cogoni, C., and Macino, G. (1999) Nature 399 (6732), 166-9.
Dunn, S. J., Park, S. W., Sharma, V., Raghu, G., Simone, J. M., Tavassoli, R., Young, L. M., Ortega, M. A., Pan, C-H., Alegre, G. J., Roninson, I. B., Lipkina, G., Dayn, A., and Holzmayer, T. A. (1999) Gene Therapy 6, 130-137.
Elbashir, S. M., Lendeckel, W., and Tuschl, T. (2001) Genes & Dev 15:188-200.
Elbashir, S. M., Harborth, J., Lendeckel, W., Yalcin, A., Weber, K., and Tuschl, T. (2001) Nature 411(6836), 494-8.
Fire, A., Xu, S., Montgomery, M. K., Kostas, S. A., Driver, S. E., and Mello, C. C. (1998) Nature 391(6669), 806-11.
Hammond, S. M., Bernstein, E., Beach, D., and Hannon, G. J. (2000) Nature 404: 293-96.
Hammond, S. M., Caudy, A. A., and Hannon, G. J. (2001) Nat. Rev. Genet. 2, 110-19.
Hofacker et al. (1994) Monatshefte F. Chemie 125:167-188.
Hsu, J. Y., Reimann, J. D. R., Sorensen, C. S., Lucas, J., and Jackson, P. K. (2002) Nature Cell Biol. 4, 358-366.
Hutvagner et al, A cellular function for the RNA-interference enzyme dicer in the maturation of the let-7 small temporal RNA. (2001)Science 293: 834-838.
Kawasaki, H., Onuki, R., Suyama, E. and Taira, K. (2002) Nature Biotech 20, 376-380
Kennerdell, J. R., and Carthew, R. W. (1998) Cell 95(7), 1017-26.
Kumar, M., and Carmichael, G. G. (1998) Microbiol Mol Biol Rev 62(4), 1415-34.
Lagos-Quintana, M., Rauhut, R., Lendeckel, W., and Tuschl, T. (2001) Science 294, 853-858.
Lau, N. C., Lim, L. P., Weinstein, E. G., and Bartel, D. P. (2001) Science 294, 858-862.
Lee, R. C. and Ambros, V. (2001) Science 294, 862-864.
Lohmann, J. U., Endl, I., and Bosch, T. C. (1999) Dev Biol 214(1), 211-4.
Maniatis T. et al., (1987) Science 236:1237.
Maxam and Gilbert (1980) in Grossman and Moldave (eds.) Academic Press, New York, Methods in Enzymology 65:499-560.
McCaskill (1990) Biopolymers 29:1105-1119.
Needham-VanDevanter et al (1984) Nucleic Acids Res., 12:6159-6168.
Ngo, H., Tschudi, C., Gull, K., and Ullu, E. (1998) Proc Natl Acad Sci USA 95(25), 14687-92.
Paddison, P., Caudy, A. A., and Hannon, G. J. (2002) Proc. Natl Acad. Sci. 99, 1443-1448.
Paddison, P. J., Caudy, A. A., Bernstein, E., Hannon, G. J., and Conklin, D. S. (2002) Genes & Dev 16, 948-958.
Paul, C. P., Good, P. D., Winer, I, and Engelke, D. R. (2002) Nature Biotech 20, 505-508.
Pearson and Regnier (1983) J. Chrom. 255:137-149.
Raponi, M., Dawes, and Arndt, G. M. Characterization of flanking sequences using long inverse PCR. (2000) Biotechniques 28, 840-844.
Sambrook et al (1989) Molecular Cloning, A laboratory Manual, Cold Spring Harbor Press, Plainview N.Y.
Schoppmeier, M., and Damen, W. G. (2001) Dev Genes Evol 211(2), 76-82.
Sui, G., Soohoo, C., Affar, E., Gay, F., Shi, Y., Forrester, W. C., and Shi, Y. (2002) Proc Natl Acad Sci 99, 5515-20.
Svoboda, P., Stein, P., Hayashi, H., and Schultz, R. M. (2000) Development 127(19), 4147-4156.
Tavernarakis, N., Wang, S. L., Dorovkov, M., Ryazanov, A., and Driscoll, M. (2000) Nat Genet 24(2), 180-3.
Thompson, B., Tonwsley, F., Rosin-Arbesfeld, R., Muisi, H., and Bienz, M. (2002) Nature Cell Biol. 4, 367-373.
Wargelius, A., Ellingsen, S., and Fjose, A. (1999) Biochem Biophys Res Commun 263(1), 156-61.
Wianny, F., and Zernicka-Goetz, M. (2000) Nat Cell Biol 2(2), 70-5.
Xu, X., Leo, C., Jang, Y., Chan, E., Padilla, D., Huang, B. C. B., Lin, T., Gururaja, T., Hitoshi, Y., Lorens, J. B., Anderson, D. C., Sikic, B., Luo, Y., Payan, D. G. and Nolan, G. P. (2001) Nature Genetics 21:23-29.
Yang, S., Tutton, S., Pierce, E., and Yoon, K. (2001) Mol Cell Biol 21(22), 7807-16.
Yu, J., DeRuiter, S. L., and Turner, D. L. (2002) Proc Nail Acad Sci 99, 6047-52.

1. A method of producing a DNA molecule wherein mRNA transcribed from the DNA molecule forms hairpin RNA (hRNA), the method comprising:
(i) synthesizing a first DNA strand comprising in order a first sequence, a random sequence and a second sequence, wherein nucleotides at the 3′ end of the second sequence are complementary to nucleotides at the 5′ end of the second sequence such that the second sequence forms a stem loop;
(ii) synthesizing a complementary DNA strand extending from the stem loop using a DNA polymerase, the complementary DNA strand being complementary to the first sequence and the random sequence so as to form hairpin DNA;
(iii) denaturing the hairpin DNA to form a single DNA strand; and
(iv) adding a primer which hybridises to the complement of the first sequence and DNA polymerase to synthesize double stranded DNA.

2. A method according to claim 1 wherein a deoxyuracil nucleotide is included in the first sequence and prior to addition of the primer the single DNA strand is depurinated and β-eliminated.

3. A method according to claim 1, wherein the first DNA strand includes a restriction enzyme site.

4. A method according to claim 1, wherein the random sequence is at least 19 base pairs, preferably 19 to about 30 base pairs, more preferably from 19 to 25 base pairs in length.

5. A method according to claim 4 wherein the random sequence is 19 base pairs in length.

6. A method according to claim 1, wherein the double stranded DNA is cloned into an expression vector under the control of a U6 snRNA, H1 or T7 promoter.

7. A method according to claim 6 wherein the double stranded DNA is cloned into an expression vector under the control of a U6 snRNA promoter.

8. A method of preparing an expression vector, expression of which produces double stranded RNA (dsRNA), the method comprising:
(i) synthesizing a first DNA strand comprising in order at least four consecutive adenosine nucleotides, a random sequence, at least four consecutive thymidine nucleotides and a primer binding site;
(ii) annealing a primer to the primer binding site and synthesizing a second DNA strand which is substantially complementary to the first DNA strand and forms double stranded DNA; and
(iii) cloning the double stranded DNA into an expression vector between two convergent promoters.

9. A method according to claim 8 wherein the random sequence is at least 19 base pairs, preferably 19 to about 30 base pairs, more preferably from 19 to 25 base pairs in length.

10. A method according to claim 9 wherein the random sequence is 19 base pairs in length.

11. A method according to claim 8, wherein the double stranded DNA is cloned into an expression vector under the control of a U6 snRNA, H1 or T7 promoter.

12. A method according to claim 11 wherein the double stranded DNA is cloned into an expression vector between two convergent U6 snRNA promoters.

13. A method for determining a function of a gene, the method comprising:
(i) synthesizing a first DNA strand comprising in order a first sequence, a random sequence and a second sequence, wherein nucleotides at the 3′ end of the second sequence are complementary to nucleotides at the 5′ end of the second sequence such that the second sequence forms a stem loop;
(ii) synthesizing a complementary DNA strand extending from the stem loop using a DNA polymerase, the complementary DNA strand being complementary to the first region and the random sequence so as to form hairpin DNA;
(iii) denaturing the hairpin DNA to form a single DNA strand;
(iv) adding a primer which hybridises to the complement of the first sequence and DNA polymerase to synthesize double stranded DNA;
(v) cloning the double stranded DNA into an expression vector wherein the double stranded DNA is under the control of a promoter;
(vi) transfecting an effective amount of the expression vector into a cell under conditions permitting transcription of the double stranded DNA to produce a transfected cell; and
(vii) detecting one or more changes in the transfected cell relative to a control cell.

14. A method according to claim 13 wherein a deoxyuracil nucleotide is included in the first sequence and prior to addition of the primer the single DNA strand is depurinated and β-eliminated.

15. A method according to claim 13, wherein the first DNA strand includes a restriction enzyme site.

16. A method according to claim 13, wherein the random sequence is at least 19 base pairs, preferably 19 to about 30 base pairs, more preferably from 19 to 25 base pairs in length.

17. A method according to claim 16 wherein the random sequence is 19 base pairs in length.

18. A method according to claim 13, wherein the double stranded DNA is cloned into an expression vector under the control of a U6 snRNA, H1 or T7 promoter.

19. A method according to claim 18 wherein the double stranded DNA is cloned into an expression vector under the control of a U6 snRNA promoter.

20. A method for determining a function of a gene, the method comprising:
(i) synthesizing a first DNA strand comprising in order at least four consecutive adenosine nucleotides, a random sequence, at least four consecutive thymidine nucleotides and a primer binding site;
(ii) annealing a primer to the primer binding site and synthesizing a second DNA strand which is substantially complementary to the first DNA strand and forms double stranded DNA;
(iii) cloning the double stranded DNA into an expression vector between two convergent promoters;
(iv) transfecting an effective amount of the expression vector into a cell under conditions favouring transcription of the double stranded DNA to produce a transfected cell; and
(v) detecting one or more changes in the transfected cell relative to a control cell.

21. A method according to claim 20 wherein the random sequence is about 19 to about 30 base pairs in length, preferably from 19 to 25 base pairs in length.

22. A method according to claim 21 wherein the random sequence is 19 base pairs in length.

23. A method according to claim 20, wherein the double stranded DNA is cloned into an expression vector under the control of a U6 snRNA, H1 or T7 promoter.

24. A method according to claim 23 wherein the double stranded DNA is cloned into an expression vector between two convergent U6 snRNA promoters.

25. An expression vector for use in suppressing expression of a target gene, the vector comprising a pair of convergent promoters and a DNA molecule positioned therebetween, the DNA molecule comprising a target-specific sequence flanked by two directional transcription terminators, the target-specific sequence comprising a sequence of at least 14 nucleotides having at least 90% identity to a segment of the target gene.

26. An expression vector according to claim 25 wherein the target-specific sequence is at least 19 base pairs, preferably 19 to about 30 base pairs, more preferably from 19 to 25 base pairs in length.

27. An expression vector according to claim 26 wherein the target-specific sequence is 19 base pairs in length.

28. An expression vector according to claim 25, wherein the target-specific sequence has a least 95% identity, and more preferably is identical, to a segment of the target gene.

29. An expression vector according to claim 25, wherein the expression vector is a retroviral expression vector.

30. An expression vector according to claim 25, wherein the convergent promoters are U6 snRNA, H1 or T7 promoters.

31. An expression vector according to claim 25, wherein the convergent promoters are U6 snRNA promoters.

32. A method for determining a function of a target gene, the method comprising:
(i) preparing an expression vector comprising a pair of convergent promoters and a DNA molecule positioned therebetween, the DNA molecule comprising a target-specific sequence flanked by two directional transcription terminators, the target-specific sequence comprising a sequence of at least 14 nucleotides having at least 90% identity to a segment of the target gene;
(ii) transfecting an effective amount of the siRNA expression vector into a cell to produce a transfected cell; and
(iii) detecting one or more phenotypic changes in the transfected cell relative to a control cell.

33. A method according to claim 32 wherein the target-specific sequence is at least 19 base pairs, preferably 19 to about 30 base pairs, more preferably from 19 to 25 base pairs in length.

34. A method according to claim 33 wherein the target-specific sequence is 19 base pairs in length.

35. A method according to claim 32, wherein the target-specific sequence has at least 95% identity, and more preferably is identical, to a segment of the target gene.

36. A method according to claim 32, wherein the expression vector is a retroviral expression vector.

37. A method according to claim 32, wherein the convergent promoters are U6 snRNA, H1 or T7 promoters.

38. A method according to claim 32, wherein the convergent promoters are U6 snRNA promoters.

39. A method of inhibiting expression of a target gene in a cell, the method comprising introducing into the cell an expression vector according to claim 25.

40. A method of producing a library of DNA molecules wherein mRNA transcribed from the DNA molecules forms hairpin RNA (hRNA) molecules, the method comprising:
(i) preparing a library of double stranded DNA fragments;
(ii) ligating hairpin DNA to the DNA fragments from step (i);
(iii) ligating a double stranded DNA adaptor to the DNA from step (ii), wherein the DNA adaptor includes a primer binding site;
(iv) denaturing the DNA from step (iii) to form a library of single DNA strands; and
(v) adding a primer which hybridises to the primer binding site and DNA polymerase to synthesize double stranded DNA thereby producing a library of double stranded DNA molecules.

41. A method according to claim 40 wherein the library of double stranded DNA fragments is prepared by digestion of DNA.

42. A method according to claim 41 wherein the DNA is a gene.

43. A method according to claim 41 wherein the DNA is a genome.

44. A method according to claim 41 wherein the DNA is a cDNA library.

45. A method according to claim 41, wherein the digestion is with DNAseI.

46. A method according to claim 40, wherein the double stranded DNA molecules are cloned into expression vectors under the control of a promoter selected from the group consisting of U6 snRNA, H1 and T7.

47. A method according to claim 46 wherein the double stranded DNA molecules are cloned into expression vectors under the control of a U6 snRNA promoter.

48. A method of preparing a library of expression vectors, expression of which produces double stranded RNA (dsRNA) molecules, the method comprising:
(i) preparing a library of double stranded DNA fragments;
(ii) ligating a double stranded DNA adaptor to each end of the DNA fragments from step (i), wherein the sequence of the DNA adaptor comprises at least four consecutive adenosine nucleotides at the 3′ end; and
(iii) cloning the double stranded DNA from step (ii) into an expression vector between two convergent promoters.

49. A method according to claim 48 wherein the library of double stranded DNA fragments is prepared by digestion of DNA.

50. A method according to claim 49 wherein the DNA is a gene.

51. A method according to claim 49 wherein the DNA is a genome.

52. A method according to claim 49 wherein the DNA is a cDNA library.

53. A method according to claim 49, wherein the digestion is with DNAseI.

54. A method according to claim 48, wherein the double stranded DNA molecules are cloned into expression vectors under the control of a promoter selected from the group consisting of U6 snRNA, H1 and T7.

55. A method according to claim 54 wherein the double stranded DNA molecules are cloned into expression vectors under the control of a U6 snRNA promoter.

56. A method of producing a library of DNA molecules wherein mRNA transcribed from the DNA molecules forms hairpin (hRNA) molecules, the method comprising:
(i) preparing a pool of mRNA;
(ii) adding an enzyme to the pool of mRNA, wherein the enzyme reverse transcribes the mRNA to form cDNA and degrades the mRNA;
(iii) allowing the cDNA from step (ii) to form a hairpin loop;
(iv) synthesizing a second strand using the hairpin loop as a priming point for reverse transcriptase;
(v) denaturing the DNA from step (iv) to form single stranded DNA; and
(vi) adding DNA polymerase to synthesize double stranded DNA thereby producing a library of double stranded DNA molecules.

57. A method according to claim 56 wherein the enzyme of step (ii) is AMV reverse transcriptase.

58. A method according to claim 56, wherein the double stranded DNA molecules are cloned into expression vectors under the control of a promoter selected from the group consisting of U6 snRNA, H1 and T7.

59. A method according to claim 58 wherein the double stranded DNA molecules are cloned into expression vectors under the control of a U6 snRNA promoter.


SEQUENCE LISTING (41 sequences)

SEQ ID NO: 1 - DNA, 58 bp, Artificial Sequence
  Description of Combined DNA/RNA Molecule Synthetic oligonucleotide
tgtggtgattcgtcgacugactccagtggtaatctacgtcgagtctcttgaactcgac

SEQ ID NO: 2 - DNA, 17 bp, Artificial Sequence
  Description of Artificial Sequence Synthetic oligonucleotide
tgtggtgattcgtcgac

SEQ ID NO: 3 - DNA, 19 bp, Homo sapiens
gactccagtggtaatctac

SEQ ID NO: 4 - DNA, 21 bp, Artificial Sequence
  Description of Artificial Sequence Synthetic oligonucleotide
gtcgagtctcttgaactcgac

SEQ ID NO: 5 - DNA, 58 bp, Artificial Sequence
  Description of Combined DNA/RNA Molecule Synthetic oligonucleotide
tgtggtgattcgtcgacunnnnnnnnnnnnnnnnnnngtcgagtctcttgaactcgac

SEQ ID NO: 6 - DNA, 52 bp, Artificial Sequence
  Description of Artificial Sequence Synthetic oligonucleotide
tcgaccggcaagctgaccctgaagttcgcttcagggtcagcttgccgttttt

SEQ ID NO: 7 - DNA, 51 bp, Artificial Sequence
  Description of Artificial Sequence Synthetic oligonucleotide
ctagaaaaacggcaagctgaccctgaagcgaacttcagggtcagcttgccg

SEQ ID NO: 8 - DNA, 31 bp, Artificial Sequence
  Description of Artificial Sequence Synthetic oligonucleotide
gcgcctcgagatagggaattcgagctcggta

SEQ ID NO: 9 - DNA, 30 bp, Artificial Sequence
  Description of Artificial Sequence Synthetic oligonucleotide
gcgcggatccttgtaaacgacggccagtgc

SEQ ID NO: 10 - DNA, 45 bp, Artificial Sequence
  Description of Artificial Sequence Synthetic oligonucleotide
tcgactcaagttatacccttgccgatagactgcttacatttaaat

SEQ ID NO: 11 - DNA, 45 bp, Artificial Sequence
  Description of Artificial Sequence Synthetic oligonucleotide
ctagatttaaatgtaagcagtctatcggcaagggtataacttgag

SEQ ID NO: 12 - DNA, 63 bp, Artificial Sequence
  Description of Artificial Sequence Synthetic oligonucleotide
cggtgattccgtcgaccaaaaagactccagtggtaatctactttttctagaggtaacaggcgc

SEQ ID NO: 13 - DNA, 17 bp, Artificial Sequence
  Description of Artificial Sequence Synthetic oligonucleotide
gcgcctgttacctctag

SEQ ID NO: 14 - DNA, 34 bp, Artificial Sequence
  Description of Artificial Sequence Synthetic oligonucleotide
gcctgcaggatatttgcatgtcgctatgttctgg

SEQ ID NO: 15 - DNA, 34 bp, Artificial Sequence
  Description of Artificial Sequence Synthetic oligonucleotide
gctctagagagtggtctcatacagaacttataag

SEQ ID NO: 16 - DNA, 34 bp, Artificial Sequence
  Description of Artificial Sequence Synthetic oligonucleotide
tcgacaaaaacggcaagctgaccctgaagttttt

SEQ ID NO: 17 - DNA, 35 bp, Artificial Sequence
  Description of Artificial Sequence Synthetic oligonucleotide
ctcagaaaaacttcagggtcagcttgccgtttttg

SEQ ID NO: 18 - DNA, 63 bp, Artificial Sequence
  Description of Artificial Sequence Synthetic oligonucleotide
cggtgattccctcgagcaaaaannnnnnnnnnnnnnnnnnntttttctagaggtaacaggcgc

SEQ ID NO: 19 - DNA, 31 bp, Artificial Sequence
  Description of Artificial Sequence Synthetic oligonucleotide
gcgcaagcttatagggaattcgagctcggta

SEQ ID NO: 20 - DNA, 31 bp, Artificial Sequence
  Description of Artificial Sequence Synthetic oligonucleotide
gcgctctagaggtgtttcgtcctttccacaa

SEQ ID NO: 21 - DNA, 34 bp, Artificial Sequence
  Description of Artificial Sequence Synthetic oligonucleotide
ctagaaaaacttcagggtcagcttgccgtttttg

SEQ ID NO: 22 - DNA, 34 bp, Artificial Sequence
  Description of Artificial Sequence Synthetic oligonucleotide
tcgacaaaaagactccagtggtaatctacttttt

SEQ ID NO: 23 - DNA, 34 bp, Artificial Sequence
  Description of Artificial Sequence Synthetic oligonucleotide
ctagaaaaagtagattaccactggagtctttttg

SEQ ID NO: 24 - DNA, 21 bp, Artificial Sequence
  Description of Combined DNA/RNA Molecule Synthetic oligonucleotide
cggcaagcugacccugaagtt

SEQ ID NO: 25 - DNA, 21 bp, Artificial Sequence
  Description of Combined DNA/RNA Molecule Synthetic oligonucleotide
gacuccagugguaaucuactt

SEQ ID NO: 26 - DNA, 21 bp, Artificial Sequence
  Description of Combined DNA/RNA Molecule Synthetic oligonucleotide
gcaugaaccggaggcccautt

SEQ ID NO: 27 - DNA, 32 bp, Artificial Sequence
  Description of Artificial Sequence Synthetic oligonucleotide
gcgctagccgttaactcgaggatccaaggtcg

SEQ ID NO: 28 - DNA, 32 bp, Artificial Sequence
  Description of Artificial Sequence Synthetic oligonucleotide
gcgctagccacagccggatccttgtaaacgac

SEQ ID NO: 29 - DNA, 33 bp, Artificial Sequence
  Description of Artificial Sequence Synthetic oligonucleotide
gcgctcgagcacagccggatccttgtaaacgac

SEQ ID NO: 30 - DNA, 29 bp, Artificial Sequence
  Description of Artificial Sequence Synthetic oligonucleotide
gcagtcgacggtaccgcgggcccggtcgc

SEQ ID NO: 31 - DNA, 29 bp, Artificial Sequence
  Description of Artificial Sequence Synthetic oligonucleotide
ggaattcgcggccgctttacttgtacagc

SEQ ID NO: 32 - DNA, 15 bp, Artificial Sequence
  Description of Artificial Sequence Synthetic oligonucleotide
gtcgacgggtctaga

SEQ ID NO: 33 - DNA, 65 bp, Artificial Sequence
  Description of Artificial Sequence Synthetic oligonucleotide
tcgactnnnnnnnnnnnnnnnnnnnnnnnnnnnnnnnnnnnnnnnnnnnnnnnnnnnnnnnnnnn

SEQ ID NO: 34 - DNA, 65 bp, Artificial Sequence; identical to SEQ ID NO: 33
  Description of Artificial Sequence Synthetic oligonucleotide

SEQ ID NO: 35 - DNA, 51 bp, Artificial Sequence
  Description of Artificial Sequence Synthetic oligonucleotide
gtcgactcaagttatacccttgccgatagactgcttacatttaaatctaga

SEQ ID NO: 36 - DNA, 17 bp, Artificial Sequence
  Description of Artificial Sequence Synthetic oligonucleotide
gcgcctgttacctctag

SEQ ID NO: 37 - DNA, 35 bp, Artificial Sequence
  Description of Artificial Sequence Synthetic oligonucleotide
tcgaccaaaaagactccagtggtaatctacttttt

SEQ ID NO: 38 - DNA, 35 bp, Artificial Sequence
  Description of Artificial Sequence Synthetic oligonucleotide
ctagaaaaagtagattaccactggagtctttttgg

SEQ ID NO: 39 - DNA, 35 bp, Artificial Sequence
  Description of Artificial Sequence Synthetic oligonucleotide
tcgaccaaaaannnnnnnnnnnnnnnnnnnttttt

SEQ ID NO: 40 - DNA, 35 bp, Artificial Sequence
  Description of Artificial Sequence Synthetic oligonucleotide
ctagaaaaannnnnnnnnnnnnnnnnnntttttgg

SEQ ID NO: 41 - DNA, 29 bp, Artificial Sequence
  Description of Artificial Sequence Synthetic oligonucleotide
aaaaannnnnnnnnnnnnnnnnnnttttt